(19) United States
(12) Patent Application Publication
(10) Pub. No.: US 2012/0053200 A1
(43) Pub. Date: Mar. 1, 2012

(54) BACE 2 INHIBITORS

(76) Inventors: Harald Mauser, Birsfelden (CH); Matthias Nettekoven, Grenzach-Wyhlen (DE); Sébastien Schmitt, Hagenthal-le-Bas (FR)

(21) Appl. No.: 13/215,273

(22) Filed: Aug. 23, 2011

(30) Foreign Application Priority Data
Sep. 1, 2010 (EP) 10174819.2

(51) Int. Cl. A61K 31/439; A61K 31/4439; C07D 207/06; A61K 31/40; C07D 207/08; C07D 409/04; A61K 31/4025; C07D 213/56; A61K 31/4402; C07D 417/04; A61K 31/427; C07D 277/30; A61K 31/426; C07D 409/14; A61K 31/4155; C07D 221/22; A61P 3/10; C07D 417/12

(52) U.S. Cl. 514/299; 546/269.7; 514/342; 548/539; 514/423; 548/527; 514/422; 546/337; 514/357; 548/204; 514/365; 548/365.7; 514/406; 514/341; 546/112

(57) ABSTRACT

The present invention relates to N-1-Benzyl-2-hydroxy-3-(hetero)arylamino-propyl)-isophthalamides of formula I

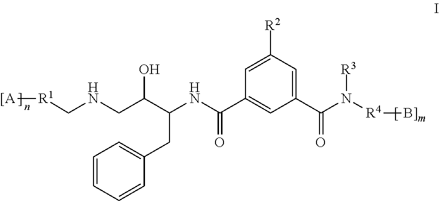

having BACE2 inhibitory activity and their use as therapeutically active substances, their manufacture and pharmaceutical compositions. The active compounds of the present invention are useful in the therapeutic and/or prophylactic treatment of e.g. type 2 diabetes.

### PRIORITY TO RELATED APPLICATION(S)

This application claims the benefit of European Patent Application No. 10174819.2, filed Sep. 1, 2010, which is hereby incorporated by reference in its entirety.

### FIELD OF THE INVENTION

The present invention is concerned with N-1-Benzyl-2-hydroxy-3-arylamino-propyl)-isophthalamides and N-1-Benzyl-2-hydroxy-3-heteroarylamino-propyl)-isophthalamides having BACE2 inhibitory properties, their manufacture, pharmaceutical compositions containing them and their use as therapeutically active substances.

The compounds may be used in the therapeutic and/or prophylactic treatment of diseases and disorders such as type 2 diabetes and other metabolic disorders.

### BACKGROUND OF THE INVENTION

Type 2 diabetes (T2D) is caused by insulin resistance and inadequate insulin secretion from pancreatic β-cells leading to poor blood-glucose control and hyperglycemia (M Prentki & C J Nolan, “Islet beta-cell failure in type 2 diabetes.” J. Clin. Investig. 2006, 116(7), 1802-1812). Patients with T2D have an increased risk of microvascular and macrovascular disease and a range of related complications including diabetic nephropathy, retinopathy and cardiovascular disease. In 2000, an estimated 171 million people had the condition with the expectation that this FIGURE will double by 2030 (S Wild, G Roglic, A Green, R. Sicree & H King, “Global prevalence of diabetes”, Diabetes Care 2004, 27(5), 1047-1053), making the disease a major healthcare problem. The rise in prevalence of T2D is associated with an increasingly sedentary lifestyle and high-energy food intake of the world's population (P Zimmet, K G M M Alberti & J Shaw, “Global and societal implications of the diabetes epidemic” Nature 2001, 414, 782-787).

β-Cell failure and consequent dramatic decline in insulin secretion and hyperglycemia marks the onset of T2D. Most current treatments do not prevent the loss of β-cell mass characterizing overt T2D. However, recent developments with GLP-1 analogues, gastrin and other agents show that preservation and proliferation of 13-cells is possible to achieve, leading to an improved glucose tolerance and slower progression to overt T2D (L L Baggio & D J Drucker, “Therapeutic approaches to preserve islet mass in type 2 diabetes”, Annu Rev. Med. 2006, 57, 265-281).

Tmem27 has been identified as a protein promoting beta-cell proliferation (P Akpinar, S Kuwajima, J Kriitzfeldt, M Stoffel, “Tmem27: A cleaved and shed plasma membrane protein that stimulates pancreatic β cell proliferation”, Cell Metab. 2005, 2, 385-397) and insulin secretion (K Fukui, Q Yang, Y Cao, N Takahashi et al., “The HNF-1 target Collectrin controls insulin exocytosis by SNARE complex formation”, Cell Metab. 2005, 2, 373-384). Tmem27 is a 42 kDa membrane glycoprotein which is constitutively shed from the surface of 13-cells, resulting from a degradation of the full-length cellular Tmem27. Overexpression of Tmem27 in a transgenic mouse increases β-cell mass and improves glucose tolerance in a diet-induced obesity (DIO) model of diabetes. Furthermore, siRNA knockout of Tmem27 in a rodent β-cell proliferation assay (e.g. using INS1e cells) reduces the proliferation rate, indicating a role for Tmem27 in control of β-cell mass.

In the same proliferation assay, BACE2 inhibitors also increase proliferation. However, BACE2 inhibition combined with Tmem27 siRNA knockdown results in low proliferation rates. Therefore, it is concluded that BACE2 is the protease responsible for the degradation of Tmem27. Furthermore, in vitro, BACE2 cleaves a peptide based on the sequence of Tmem27. The closely related protease BACE1 does not cleave this peptide and selective inhibition of BACE1 alone does not enhance proliferation of β-cells.

The close homolog BACE2 is a membrane-bound aspartyl protease and is co-localized with Tmem27 in human pancreatic β-cells (G Finzi, F Franzi, C Placidi, F Acquati et al., “BACE2 is stored in secretory granules of mouse and rat pancreatic beta cells”, Ultrastruct Pathol. 2008, 32(6), 246-251). It is also known to be capable of degrading APP (I Hussain, D Powell, D Howlett, G Chapman et al., “ASP1 (BACE2) cleaves the amyloid precursor protein at the β-secretase site” Mol Cell Neurosci. 2000, 16, 609-619), IL-1R2 (P Kuhn, E Marjaux, A Imhof, B De Strooper et al., “Regulated intramembrane proteolysis of the interleukin-1 receptor II by alpha-, beta-, and gamma-secretase” J. Biol. Chem. 2007, 282(16), 11982-11995) and ACE2. The capability to degrade ACE2 indicates a possible role of BACE2 in the control of hypertension.

Inhibition of BACE2 is therefore proposed as a treatment for T2D with the potential to preserve and restore β-cell mass and stimulate insulin secretion in pre-diabetic and diabetic patients. It is therefore an object of the present invention to provide selective BACE2 inhibitors. Such compounds are useful as therapeutically active substances, particularly in the treatment and/or prevention of diseases which are associated with the inhibition of BACE2.

### DETAILED DESCRIPTION OF THE INVENTION

The following definitions of the general terms used in the present description apply irrespectively of whether the terms in question appear alone or in combination with other groups.

The term “C₁₋₆alkyl”, alone or in combination with other groups, stands for a hydrocarbon radical which can be linear or branched, with single or multiple branching, for example, methyl (Me), ethyl (Et), propyl, isopropyl (i-propyl), n-butyl, i-butyl (iso-butyl), 2-butyl (sec-butyl), t-butyl (tert-butyl) and the like. Particular are groups with 1 to 4 carbon atoms, for example methyl, ethyl and isopropyl. Specific is methyl.

The term “halogen-C₁₋₆alkyl”, alone or in combination with other groups, refers to C₁₋₆alkyl, which is substituted by one or multiple halogens, in particular F. Particular are trifluoro-C₁₋₆alkyl, halogen-methyl and halogen-ethyl. Specific are trifluoro-methyl and 1-fluoro-1-methyl-ethyl.

The term “acetyl”, alone or in combination with other groups, refers to —C(═O)—CH₃.

The term “acetamidyl”, alone or in combination with other groups, refers to —C(═NH)—NH₂.

The term “amido”, alone or in combination with other groups, refers to —C(═O)—NH₂.

The term “amino”, alone or in combination with other groups, refers to —NH₂.

The term “benzoyl”, alone or in combination with other groups, refers to —C(═O)-phenyl.

The term “benzyl”, alone or in combination with other groups, refers to —CH₂-phenyl.

The term “carboxy”, alone or in combination with other groups, refers to —C(═O)OH.

The term “cyano”, alone or in combination with other groups, refers to N≡C— (NC—).

The term “hydroxy”, alone or in combination with other groups, refers to —OH.

The term “nitro”, alone or in combination with other groups, refers to —NO₂.

The term “methanesulfonyl”, alone or in combination with other groups, refers to —SO₂—CH₃.

The term “halogen”, alone or in combination with other groups, denotes chloro (Cl), iodo (I), fluoro (F) and bromo (Br). Particular “halogen” is chloro and fluoro. Specific is fluoro.

The term “aryl”, alone or in combination with other groups, refers to an aromatic carbocyclic group comprising 6 to 14, preferably 6 to 10, carbon atoms and having at least one aromatic ring or multiple condensed rings in which at least one ring is aromatic. Examples of “aryl” include biphenyl, indanyl, naphthyl, phenyl (Ph) and the like. Particular is one aromatic ring having 6 to 10 carbon atoms. Specific is phenyl.

The term “heteroaryl”, alone or in combination with other groups, refers to an aromatic carbocyclic group of having a single 5 to 8 membered ring or multiple condensed rings comprising 6 to 14, more preferably 6 to 10, ring atoms and containing 1, 2 or 3 heteroatoms, in which group at least one heterocyclic ring is aromatic and the heteroatoms are individually selected from O, S and N. Examples of “heteroaryl” include benzofuryl, benzoimidazolyl, benzooxazinyl, benzothiazinyl, benzothiazolyl, benzothienyl, benzotriazolyl, furyl, imidazolyl, indazolyl, indolyl, isoquinolinyl, isothiazolyl, isoxazolyl, oxazolyl, pyrazinyl, pyrazolyl (pyrazyl), pyrazolo[1,5-a]pyridinyl, pyridazinyl, pyridinyl, pyrimidinyl, pyrrolyl, quinolinyl, tetrazolyl, thiazolyl, thienyl, triazolyl and the like. Particular “heteroaryl” have a single 5 to 8 membered ring. Specific are [1,2,4]oxadiazolyl, 1H-pyrazolyl, 2H-pyrazolyl, isoxazolyl, pyridinyl, thiazolyl and thiophenyl. More specific are [1,2,4]oxadiazol-5-yl, 1H-pyrazol-3-yl, 2H-pyrazol-3-yl, isoxazol-5-yl, pyridin-2-yl, pyridin-3-yl, pyridin-4-yl, thiazol-2-yl and thiophen-2-yl.

The term “cycloalkyl”, alone or in combination with other groups, refers to a 3 to 6 membered carbon ring, for example cyclopropyl, cyclobutyl, cyclopentyl or cyclohexyl. Particular have a 5 or 6 membered carbon ring. Specific are cyclopropyl and cyclohexyl.

The term “C₁₋₆alkoxy”, alone or in combination with other groups, stands for an —O—C₁₋₆alkyl radical, which C₁₋₆alkyl can be linear or branched, with single or multiple branching, for example, methoxy (OMe, MeO), ethoxy (OEt), propoxy, isopropoxy (i-propoxy), n-butoxy, i-butoxy (iso-butoxy), 2-butoxy (sec-butoxy), t-butoxy (tert-butoxy), isopentyloxy (i-pentyloxy) and the like. Particular are groups with 1 to 4 carbon atoms. Specific is methoxy.

The term “halogen-C₁₋₆alkoxy”, alone or in combination with other groups, refers to C₁₋₆alkoxy, which is substituted by one or multiple halogen, in particular F. Particular “halogen-C₁₋₆alkoxy” are fluoro-lower alkoxy, fluoro-methoxy and halogen-methoxy. Specific is trifluoro methoxy.

The term “heterocyclyl”, alone or in combination with other groups, refers to a 4 to 8 membered ring containing 1, 2 or 3 ring heteroatoms individually selected from N, O or S. 1 or 2 ring heteroatoms are preferred. Particular are 5 to 6 membered “heterocyclyl”, each containing 1 or 2 ring heteroatoms selected from N, O or S. More particular is a five membered heterocyclyl, specific pyrrolidinyl. Examples of “heterocyclyl” include azepanyl, azetidyl, diazepanyl, morpholinyl, oxazepanyl, oxazolidyl, oxetanyl, piperazinyl, piperidyl, pyrrolidinyl, tetrahydrofuryl, tetrahydropyridyl, tetrahydropyryl, tetrahydrothienyl, thiazolidyl, thiomorpholinyl and the like. Specific is pyrrolidinyl, more specific pyrrolidin-1-yl.

The term “pharmaceutically acceptable salts” refers to salts that are suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid, sulphuric acid, tartaric acid, trifluoroacetic acid and the like. Specific are formic acid and hydrochloric acid. More specific is hydrochloric acid.

The terms “pharmaceutically acceptable carrier” and “pharmaceutically acceptable auxiliary substance” refer to carriers and auxiliary substances such as diluents or excipients that are compatible with the other ingredients of the formulation.

Substituents at a double bond or a ring can be present in cis (═Z—) or trans (=E-) form, unless the stereochemistry is explicitly depicted in the corresponding compound formula I.

The term “pharmaceutical composition” encompasses a product comprising specified ingredients in pre-determined amounts or proportions, as well as any product that results, directly or indirectly, from combining specified ingredients in specified amounts. Preferably it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

The following table lists abbreviations used within the present document.

TABLE 1
abbreviations

| | |
|---|---|
| AcOEt | ethyl acetate |
| DCM | dichloromethane |
| DIPEA | diisopropylethylamin |
| DMF | dimethylformamide |
| EDCI | 1-ethyl-3-(3-dimethylaminopropyl) carbodiimide |
| HATU | 2-(1H-7-Azabenzotriazol-1-yl)-1,1,3,3-tetramethyl |
| | uronium hexafluorophosphate |
| HCl | hydrochloric acid |
| HPLC | high performance liquid chromatography |
| KHSO₄ | potassium sulfate |
| LiOH•H₂O | lithium hydroxide monohydrate |
| MgSO₄ | magnesium sulfate |
| NaBH(OAc)₃ | sodium triacetoxyborohydride |
| NaBH₄ | sodium borohydride |
| NaHCO₃ | sodium hydrogencarbonate |
| NEt₃ | triethylamin |
| NMR | nuclear magnetic resonance |
| TBTU | O-Benzotriazole-1-yl-N,N,N′,N′-tetramethyluronium |
| | tetrafluoroborate |
| TFA | trifluoro acetic acid |
| THF | tetrahydrofuran |

The invention also provides pharmaceutical compositions, methods of using, and methods of preparing the aforementioned compounds.

All separate embodiments can be combined.

One embodiment of the invention provides a method for the treatment of diabetes comprising the step of administering to a patient in need thereof a compound of formula I,

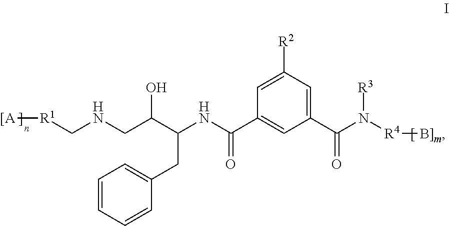

wherein
R¹ is selected from the group consisting of
i) aryl, and
ii) heteroaryl,
R² is selected from the group consisting of

### i) H,

ii) C₁₋₆-alkyl, and
iii) —N(R⁵,R⁶);
R³ is selected from the group consisting of

### i) H, and

ii) C₁₋₆-alkyl,
R⁴ is selected from the group consisting of
i) —CH₂-aryl, and
ii) —CH₂-heteroaryl;
or R³ and R⁴ together with the nitrogen to which they are attached form a five membered heterocyclyl, optionally substituted by Z; or form 3-aza-bicyclo[3.2.1]octane-3-yl, optionally substituted by C₁₋₆-alkyl;
R⁵ is selected from the group consisting of

### i) H, and

ii) C₁₋₆-alkyl;
R⁶ is —SO₂—C₁₋₆-alkyl;
n is 0, 1 or 2;
m is 0, 1 or 2;
A is independently selected from the group consisting of
i) acetamidyl,
ii) acetyl,
iii) amido,
iv) amino,
v) C₁₋₆-alkoxy,
vi) C₁₋₆-alkyl,
vii) carboxy,
viii) cyano,
ix) halogen,
x) halogen-C₁₋₆-alkoxy,
xi) halogen-C₁₋₆-alkyl,
xii) hydroxy,
xiii) —N(C₁₋₆-alkyl, C₁₋₆-alkyl),
xiv) —N(H, C₁₋₆-alkyl), and
xv) —SO₂—C₁₋₆-alkyl
B is C₁₋₆-alkyl; and
Z is independently selected from the group consisting of
i) aryl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
ii) heteroaryl, optionally substituted by 1 or 2 C₁₋₆-alkyl,
iii) cycloalkyl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
iv) acetyl,
v) benzoyl, optionally substituted by 1 or 2 substituents individually selected from acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
vi) benzyl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro, and
vii) C₁₋₆-alkyl, optionally substituted by 1 or 2 substituents individually selected from the group consisting of amino, cyano, halogen, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy and nitro,
or a pharmaceutically acceptable salt thereof.

One embodiment of the invention is a compound of formula Ia,

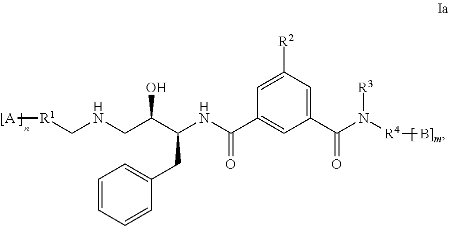

wherein
R¹ is selected from the group consisting of
i) aryl, and
ii) heteroaryl,
R² is selected from the group consisting of

### i) H,

ii) C₁₋₆-alkyl, and
iii) —N(R⁵,R⁶);
R³ is selected from the group consisting of

### i) H, and

ii) C₁₋₆-alkyl,
R⁴ is selected from the group consisting of
i) —CH₂-aryl, and
ii)-CH₂-heteroaryl;
or R³ and R⁴ together with the nitrogen to which they are attached form a five membered heterocyclyl, optionally substituted by Z; or form 3-aza-bicyclo[3.2.1]octane-3-yl, optionally substituted by C₁₋₆-alkyl;
R⁵ is selected from the group consisting of

### i) H, and

ii) C₁₋₆-alkyl;
R⁶ is —SO₂—C₁₋₆-alkyl;
n is 0, 1 or 2;
m is 0, 1 or 2;
A is independently selected from the group consisting of
i) acetamidyl,
ii) acetyl,
iii) amido,
iv) amino,
v) C₁₋₆-alkoxy,
vi) C₁₋₆-alkyl,
vii) carboxy,
viii) cyano,
ix) halogen,
x) halogen-C₁₋₆-alkoxy,
xi) halogen-C₁₋₆-alkyl,
xii) hydroxy,
xiii) —N(C₁₋₆-alkyl, C₁₋₆-alkyl), and
xiv) —N(H, C₁₋₆-alkyl), and
xv) —SO₂—C₁₋₆-alkyl
B is C₁₋₆-alkyl; and
Z is independently selected from the group consisting of
i) aryl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
ii) heteroaryl, optionally substituted by 1 or 2 C₁₋₆-alkyl,
iii) cycloalkyl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
iv) acetyl,
v) benzoyl, optionally substituted by 1 or 2 substituents individually selected from acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
vi) benzyl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro, and
vii) C₁₋₆-alkyl, optionally substituted by 1 or 2 substituents individually selected from the group consisting of amino, cyano, halogen, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy and nitro,
or a pharmaceutically acceptable salt thereof.

One embodiment of the invention is a method for the treatment of diabetes comprising the step of administering to a patient in need thereof a compound of formula I, wherein

R¹ is selected from the group consisting of
i) phenyl,
ii) pyrazolyl, and
iii) pyridinyl;
R² is selected from the group consisting of

### i) H,

ii) C₁₋₆-alkyl, and
iii) —N(R⁵,R⁶);
R³ is selected from the group consisting of

### i) H, and

ii) C₁₋₆-alkyl,
R⁴ is selected from the group consisting of
i) —CH₂-pyridinyl, and
ii)-CH₂-thiazolyl
or R³ and R⁴ together with the nitrogen to which they are attached form pyrrolidinyl, optionally substituted by Z; or form 3-aza-bicyclo[3.2.1]octane-3-yl, optionally substituted by C₁₋₆-alkyl;
R⁵ is selected from the group consisting of

### i) H, and

ii) C₁₋₆-alkyl;
R⁶ is —SO₂—C₁₋₆-alkyl;
n is 0 or 1;
m is 0, 1 or 2;
A is independently selected from the group consisting of
i) C₁₋₆-alkyl,
ii) halogen-C₁₋₆-alkyl,
iii) C₁₋₆-alkoxy, and
iv) halogen-C₁₋₆-alkoxy;
B is C₁₋₆-alkyl; and
Z is independently selected from the group consisting of
i) pyridinyl, optionally substituted by C₁₋₆-alkyl,
ii) thiazolyl, optionally substituted by C₁₋₆-alkyl,
iii) pyrazolyl, optionally substituted by C₁₋₆-alkyl,
iv) isoxazolyl, optionally substituted by C₁₋₆-alkyl,
v) [1,2,4]oxadiazol, optionally substituted by C₁₋₆-alkyl,
vi) thiophenyl, optionally substituted by C₁₋₆-alkyl,
vii) cyclopentyl, optionally substituted by C₁₋₆-alkyl
viii) cyclohexyl, optionally substituted by C₁₋₆-alkyl,
ix) benzoyl, optionally substituted by C₁₋₆-alkyl
x) benzyl, optionally substituted by 1 or 2 substituents independently selected from the group consisting of C₁₋₆-alkyl, halo and C₁₋₆-alkoxy
xi) phenyl, optionally substituted by 1 or 2 substituents independently selected from the group consisting of C₁₋₆-alkyl, halo and C₁₋₆-alkoxy; and
xii) C₁₋₆-alkyl;
or a pharmaceutically acceptable salt thereof.

One embodiment of the invention is a method for the treatment of diabetes comprising the step of administering to a patient in need thereof a compound of formula I, wherein R¹ is phenyl.

One embodiment of the invention is a method for the treatment of diabetes comprising the step of administering to a patient in need thereof a compound of formula I, wherein R² is H or N(methyl,methanesulfonyl).

One embodiment of the invention is a method for the treatment of diabetes comprising the step of administering to a patient in need thereof a compound of formula I, wherein R² is H.

One embodiment of the invention is a method for the treatment of diabetes comprising the step of administering to a patient in need thereof a compound of formula I, wherein R² is N(methyl,methanesulfonyl).

One embodiment of the invention is a method for the treatment of diabetes comprising the step of administering to a patient in need thereof a compound of formula I, wherein R³ is methyl and R⁴ is —CH₂-thiazolyl, optionally substituted by methyl.

One embodiment of the invention is a method for the treatment of diabetes comprising the step of administering to a patient in need thereof a compound of formula I, wherein R³ is methyl and R⁴ is —CH₂-thiazolyl, substituted by methyl

One embodiment of the invention is a method for the treatment of diabetes comprising the step of administering to a patient in need thereof a compound of formula I, wherein R³ and R⁴ together with the nitrogen to which they are attached form pyrrolidinyl, optionally substituted by methyl-thiazolyl, phenyl, thiophenyl, fluoro-phenyl, methyl cyclohexyl or cyclopentyl.

One embodiment of the invention is a method for the treatment of diabetes comprising the step of administering to a patient in need thereof a compound of formula I, wherein R³ and R⁴ together with the nitrogen to which they are attached form pyrrolidinyl, optionally substituted by methyl-thiazolyl, phenyl, thiophenyl or cyclopentyl.

One embodiment of the invention is a method for the treatment of diabetes comprising the step of administering to a patient in need thereof a compound of formula I, wherein R³ and R⁴ together with the nitrogen to which they are attached form pyrrolidinyl, substituted by methyl-thiazolyl.

One embodiment of the invention is a method for the treatment of diabetes comprising the step of administering to a patient in need thereof a compound of formula I, wherein R³ and R⁴ together with the nitrogen to which they are attached form pyrrolidinyl, substituted by phenyl, thiophenyl or cyclopentyl.

One embodiment of the invention is a method for the treatment of diabetes comprising the step of administering to a patient in need thereof a compound of formula I, wherein R³ and R⁴ together with the nitrogen to which they are attached form pyrrolidinyl, substituted by thiophenyl.

One embodiment of the invention is a method for the treatment of diabetes comprising the step of administering to a patient in need thereof a compound of formula I, wherein R³ and R⁴ together with the nitrogen to which they are attached form a 5 membered heterocyclyl, optionally substituted by a heteroaryl or C₁₋₆-alkyl-heteroyryl, which heteroaryl may contain 1,2 or 3 heteroatoms individually selected from N, S and O.

One embodiment of the invention is a method for the treatment of diabetes comprising the step of administering to a patient in need thereof a compound of formula I, wherein Z is selected from the group consisting of pyridinyl, thiazolyl, pyrazolyl, isoxazolyl, [1,2,4]oxadiazol, thiophenyl, cyclopentyl, cyclohexyl, benzoyl, benzyl, and phenyl; each optionally substituted by C₁₋₆-alkyl or Z is C₁₋₆-alkyl.

One embodiment of the invention is a compound of formula Ia,

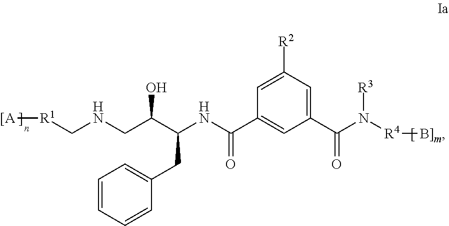

wherein the compound is selected from the group consisting of
N-[(1S,2R)-1-Benzyl-2-hydroxy-3-(3-trifluoromethoxy-benzylamino)-propyl]-3-(2-pyridin-4-yl-pyrrolidine-1-carbonyl)-benzamide,
N-[(1S,2R)-1-Benzyl-2-hydroxy-3-(3-trifluoromethyl-benzylamino)-propyl]-3-(2-pyridin-4-yl-pyrrolidine-1-carbonyl)-benzamide,
N-[(1S,2R)-1-Benzyl-2-hydroxy-3-(3-trifluoromethoxy-benzylamino)-propyl]-N′-pyridin-2-ylmethyl-isophthalamide,
N-[(1S,2R)-1-Benzyl-2-hydroxy-3-(3-trifluoromethyl-benzylamino)-propyl]-N′-pyridin-2-ylmethyl-isophthalamide,
N-[(1S,2R)-1-Benzyl-2-hydroxy-3-(3-trifluoromethoxy-benzylamino)-propyl]-3-(1R,5S)-1,8,8-trimethyl-3-aza-bicyclo[3.2.1]octane-3-carbonyl)-benzamide,
N-[(1S,2R)-1-Benzyl-2-hydroxy-3-(3-trifluoromethoxy-benzylamino)-propyl]-3-(3-pyridin-2-yl-pyrrolidine-1-carbonyl)-benzamide,
N-[(1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide,
N-[(1S,2R)-1-Benzyl-2-hydroxy-3-(3-trifluoromethyl-benzylamino)-propyl]-3-(2-phenyl-pyrrolidine-1-carbonyl)-benzamide,
N-[(1S,2R)-1-Benzyl-2-hydroxy-3-(3-trifluoromethyl-benzylamino)-propyl]-3-(2-pyridin-3-yl-pyrrolidine-1-carbonyl)-benzamide,
N-[(1S,2R)-1-Benzyl-2-hydroxy-3-(3-trifluoromethyl-benzylamino)-propyl]-3-[2-(1-methyl-1H-pyrazol-3-yl)-pyrrolidine-1-carbonyl]-benzamide,
N-[(1S,2R)-1-Benzyl-2-hydroxy-3-(3-trifluoromethyl-benzylamino)-propyl]-3-[2-(2-methyl-2H-pyrazol-3-yl)-pyrrolidine-1-carbonyl]-benzamide,
N-[(1S,2R)-1-Benzyl-2-hydroxy-3-(3-trifluoromethyl-benzylamino)-propyl]-3-[2-(3-methyl-isoxazol-5-yl)-pyrrolidine-1-carbonyl]-benzamide,
N-[(1S,2R)-1-Benzyl-2-hydroxy-3-(3-trifluoromethyl-benzylamino)-propyl]-3-[2-(3-methyl-[1,2,4]oxadiazol-5-yl)-pyrrolidine-1-carbonyl]-benzamide,
N-[(1S,2R)-1-Benzyl-2-hydroxy-3-(3-trifluoromethyl-benzylamino)-propyl]-N′-methyl-N′-pyridin-2-ylmethyl-isophthalamide,
N-[(1S,2R)-1-Benzyl-2-hydroxy-3-(3-trifluoromethyl-benzylamino)-propyl]-3-(2-thiophen-2-yl-pyrrolidine-1-carbonyl)-benzamide,
N-[(1S,2R)-1-Benzyl-2-hydroxy-3-(3-methoxy-benzylamino)-propyl]-3-[(R)-2-(4-methyl-thiazol-2-yl)-pyrrolidine-1-carbonyl]-benzamide,
N-[(1S,2R)-1-Benzyl-2-hydroxy-3-(3-trifluoromethoxy-benzylamino)-propyl]-3-[(R)-2-(4-methyl-thiazol-2-yl)-pyrrolidine-1-carbonyl]-benzamide,
N-[(1S,2R)-1-Benzyl-2-hydroxy-3-(3-methoxy-benzylamino)-propyl]-3-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(2-phenyl-pyrrolidine-1-carbonyl)-benzamide,
N-[(1S,2R)-1-Benzyl-2-hydroxy-3-(3-trifluoromethoxy-benzylamino)-propyl]-3-(2-phenyl-pyrrolidine-1-carbonyl)-benzamide,
N-[(1S,2R)-1-Benzyl-2-hydroxy-3-(3-trifluoromethoxy-benzylamino)-propyl]-3-(2-pyridin-3-yl-pyrrolidine-1-carbonyl)-benzamide,
N-[(1S,2R)-1-Benzyl-2-hydroxy-3-(3-trifluoromethoxy-benzylamino)-propyl]-3-[2-(3-methyl-isoxazol-5-yl)-pyrrolidine-1-carbonyl]-benzamide,
N-[(1S,2R)-1-Benzyl-2-hydroxy-3-(3-methoxy-benzylamino)-propyl]-3-[2-(5-methyl-thiophen-2-yl)-pyrrolidine-1-carbonyl]-benzamide,
N-[(1S,2R)-1-Benzyl-2-hydroxy-3-(3-trifluoromethoxy-benzylamino)-propyl]-N′-methyl-N′-pyridin-2-ylmethyl-isophthalamide,
N-[(1S,2R)-1-Benzyl-2-hydroxy-3-(3-methoxy-benzylamino)-propyl]-3-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-3-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-[(R)-2-(4-methyl-thiazol-2-yl)-pyrrolidine-1-carbonyl]-benzamide,
N-{(1S,2R)-1-Benzyl-3-[(1-ethyl-1H-pyrazol-4-ylmethyl)-amino]-2-hydroxy-propyl}-3-methyl-5-[(R)-2-(4-methyl-thiazol-2-yl)-pyrrolidine-1-carbonyl]-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-methyl-5-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-5,N′-dimethyl-N′-pyridin-2-ylmethyl-isophthalamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-methyl-5-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-5,N′-dimethyl-N′-(4-methyl-thiazol-2-ylmethyl)-isophthalamide,
N-[(1S,2R)-1-Benzyl-2-hydroxy-3-(3-trifluoromethoxy-benzylamino)-propyl]-3-(methanesulfonyl-methyl-amino)-5-[(R)-2-(4-methyl-thiazol-2-yl)-pyrrolidine-1-carbonyl]-benzamide,
N-[(1S,2R)-1-Benzyl-2-hydroxy-3-(3-trifluoromethoxy-benzylamino)-propyl]-3-(methanesulfonyl-methyl-amino)-5-(2-phenyl-pyrrolidine-1-carbonyl)-benzamide,
N-[(1S,2R)-1-Benzyl-2-hydroxy-3-(3-trifluoromethoxy-benzylamino)-propyl]-3-methyl-5-(2-phenyl-pyrrolidine-1-carbonyl)-benzamide,
N-[(1S,2R)-1-Benzyl-2-hydroxy-3-(3-trifluoromethoxy-benzylamino)-propyl]-5,N′-dimethyl-N′-pyridin-2-ylmethyl-isophthalamide,
N-[(1S,2R)-1-Benzyl-2-hydroxy-3-(3-trifluoromethoxy-benzylamino)-propyl]-3-(methanesulfonyl-methyl-amino)-5-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-3-methyl-5-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-5,N′-dimethyl-N′-(4-methyl-thiazol-2-ylmethyl)-isophthalamide,
N-[(1S,2R)-1-Benzyl-2-hydroxy-3-(3-trifluoromethyl-benzylamino)-propyl]-3-(methanesulfonyl-methyl-amino)-5-[(R)-2-(4-methyl-thiazol-2-yl)-pyrrolidine-1-carbonyl]-benzamide,
N-[(1S,2R)-1-Benzyl-2-hydroxy-3-(3-trifluoromethyl-benzylamino)-propyl]-3-(methanesulfonyl-methyl-amino)-5-(2-phenyl-pyrrolidine-1-carbonyl)-benzamide,
N-[(1S,2R)-1-Benzyl-2-hydroxy-3-(3-trifluoromethyl-benzylamino)-propyl]-3-methyl-5-(2-phenyl-pyrrolidine-1-carbonyl)-benzamide,
N-[(1S,2R)-1-Benzyl-2-hydroxy-3-(3-trifluoromethyl-benzylamino)-propyl]-5,N′-dimethyl-N′-pyridin-2-ylmethyl-isophthalamide,
N-[(1S,2R)-1-Benzyl-2-hydroxy-3-(3-trifluoromethyl-benzylamino)-propyl]-3-(methanesulfonyl-methyl-amino)-5-(2-thiophen-2-yl-pyrrolidine-1-carbonyl)-benzamide,
N-[(1S,2R)-1-Benzyl-2-hydroxy-3-(3-trifluoromethyl-benzylamino)-propyl]-3-methyl-5-(2-thiophen-2-yl-pyrrolidine-1-carbonyl)-benzamide,
N-[(1S,2R)-1-Benzyl-2-hydroxy-3-(3-trifluoromethyl-benzylamino)-propyl]-3-(2-p-tolyl-pyrrolidine-1-carbonyl)-benzamide,
N-[(1S,2R)-1-Benzyl-2-hydroxy-3-(3-trifluoromethyl-benzylamino)-propyl]-3-[2-(4-methoxy-phenyl)-pyrrolidine-1-carbonyl]-benzamide,
N-[(1S,2R)-1-Benzyl-2-hydroxy-3-(3-trifluoromethyl-benzylamino)-propyl]-3-[2-(3-chloro-phenyl)-pyrrolidine-1-carbonyl]-benzamide,
N-[(1S,2R)-1-Benzyl-2-hydroxy-3-(3-trifluoromethyl-benzylamino)-propyl]-3-(2-m-tolyl-pyrrolidine-1-carbonyl)-benzamide,
N-[(1S,2R)-1-Benzyl-2-hydroxy-3-(3-trifluoromethyl-benzylamino)-propyl]-3-[2-(3-chloro-4-fluoro-phenyl)-pyrrolidine-1-carbonyl]-benzamide,
N-[(1S,2R)-1-Benzyl-2-hydroxy-3-(3-trifluoromethyl-benzylamino)-propyl]-3-[2-(4-chloro-3-methyl-phenyl)-pyrrolidine-1-carbonyl]-benzamide,
N-[(1S,2R)-1-Benzyl-2-hydroxy-3-(3-trifluoromethyl-benzylamino)-propyl]-3-[2-(2,4-dimethyl-phenyl)-pyrrolidine-1-carbonyl]-benzamide,
N-[(1S,2R)-1-Benzyl-2-hydroxy-3-(3-trifluoromethyl-benzylamino)-propyl]-3-[2-(4-ethyl-phenyl)-pyrrolidine-1-carbonyl]-benzamide,
N-[(1S,2R)-1-Benzyl-2-hydroxy-3-(3-trifluoromethyl-benzylamino)-propyl]-3-[2-(4-fluoro-phenyl)-3-methyl-pyrrolidine-1-carbonyl]-benzamide,
3-[4-Acetyl-2-(4-fluoro-phenyl)-pyrrolidine-1-carbonyl]-N′-[(1S,2R)-1-benzyl-2-hydroxy-3-(3-trifluoromethyl-benzylamino)-propyl]-benzamide,
N-[(1S,2R)-1-Benzyl-2-hydroxy-3-(3-trifluoromethyl-benzylamino)-propyl]-3-((S)-2-benzyl-pyrrolidine-1-carbonyl)-benzamide,
N-[(1S,2R)-1-Benzyl-2-hydroxy-3-(3-trifluoromethyl-benzylamino)-propyl]-3-((R)-2-phenyl-pyrrolidine-1-carbonyl)-benzamide,
N-[(1S,2R)-1-Benzyl-2-hydroxy-3-(3-trifluoromethyl-benzylamino)-propyl]-3-[(S)-2-(4-fluoro-phenyl)-pyrrolidine-1-carbonyl]-benzamide,
N-[(1S,2R)-1-Benzyl-2-hydroxy-3-(3-trifluoromethyl-b enzylamino)-propyl]-3-[2-(3-fluoro-phenyl)-pyrrolidine-1-carbonyl]-benzamide,
N-[(1S,2R)-1-Benzyl-2-hydroxy-3-(3-trifluoromethyl-benzylamino)-propyl]-3-(2-cyclohexyl-pyrrolidine-1-carbonyl)-benzamide,
N-[(1S,2R)-1-Benzyl-2-hydroxy-3-(3-trifluoromethyl-benzylamino)-propyl]-3-(2-isobutyl-pyrrolidine-1-carbonyl)-benzamide,
3-(2-Benzoyl-pyrrolidine-1-carbonyl)-N′-[(1S,2R)-1-benzyl-2-hydroxy-3-(3-trifluoromethyl-benzylamino)-propyl]-benzamide,
N-[(1S,2R)-1-Benzyl-2-hydroxy-3-(3-trifluoromethyl-benzylamino)-propyl]-3-(2-cyclopentyl-pyrrolidine-1-carbonyl)-benzamide, and
N-[(1S,2R)-1-Benzyl-2-hydroxy-3-(3-trifluoromethyl-benzylamino)-propyl]-3-(2-tert-butyl-pyrrolidine-1-carbonyl)-benzamide,
   or pharmaceutically acceptable salts thereof.

One embodiment of the invention is a compound of formula Ia, wherein the compound is selected from the group consisting of
N-[(1R,1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide,
N-[(1R,1S,2R)-1-Benzyl-2-hydroxy-3-(3-trifluoromethyl-benzylamino)-propyl]-3-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-methyl-5-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-methyl-5-(2-thiophen-2-yl-pyrrolidine-1-carbonyl)-benzamide,
N-[1R,1S,2R)-1-Benzyl-2-hydroxy-3-(3-trifluoromethoxy-benzylamino)-propyl]-3-(methanesulfonyl-methyl-amino)-5-(2-phenyl-pyrrolidine-1-carbonyl)-benzamide,
N-[(1R,1S,2R)-1-Benzyl-2-hydroxy-3-(3-trifluoromethoxy-benzylamino)-propyl]-3-methyl-5-(2-phenyl-pyrrolidine-1-carbonyl)-benzamide,
N-[(1R,1S,2R)-1-Benzyl-2-hydroxy-3-(3-trifluoromethoxy-benzylamino)-propyl]-3-(methanesulfonyl-methyl-amino)-5-(2-thiophen-2-yl-pyrrolidine-1-carbonyl)-benzamide,
N-[(1R,1S,2R)-1-Benzyl-2-hydroxy-3-(3-trifluoromethoxy-benzylamino)-propyl]-3-methyl-5-(2-thiophen-2-yl-pyrrolidine-1-carbonyl)-benzamide,
N-[(1R,1S,2R)-1-Benzyl-2-hydroxy-3-(3-trifluoromethyl-benzylamino)-propyl]-3-(methanesulfonyl-methyl-amino)-5-(2-phenyl-pyrrolidine-1-carbonyl)-benzamide,
N-[(1R,1S,2R)-1-Benzyl-2-hydroxy-3-(3-trifluoromethyl-benzylamino)-propyl]-3-methyl-5-(2-thiophen-2-yl-pyrrolidine-1-carbonyl)-benzamide,
N-[(1S,2R)-1-Benzyl-2-hydroxy-3-(3-trifluoromethyl-benzylamino)-propyl]-3-[2-(4-fluoro-phenyl)-3-methyl-pyrrolidine-1-carbonyl]-benzamide,
N-[(1S,2R)-1-Benzyl-2-hydroxy-3-(3-trifluoromethyl-benzylamino)-propyl]-3-(2-cyclohexyl-pyrrolidine-1-carbonyl)-benzamide, and
N-[(1S,2R)-1-Benzyl-2-hydroxy-3-(3-trifluoromethyl-benzylamino)-propyl]-3-(2-cyclopentyl-pyrrolidine-1-carbonyl)-benzamide,
   or pharmaceutically acceptable salts thereof.

One embodiment of the invention is a compound of formula Ia, wherein the compound is selected from the group consisting of
N-[(1R,1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide,
N-[(1R,1S,2R)-1-Benzyl-2-hydroxy-3-(3-trifluoromethyl-benzylamino)-propyl]-3-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide,
N-[(1R,1S,2R)-1-Benzyl-2-hydroxy-3-(3-trifluoromethoxy-benzylamino)-propyl]-3-(methanesulfonyl-methyl-amino)-5-(2-phenyl-pyrrolidine-1-carbonyl)-benzamide,
N-[(1R,1S,2R)-1-Benzyl-2-hydroxy-3-(3-trifluoromethoxy-benzylamino)-propyl]-3-methyl-5-(2-phenyl-pyrrolidine-1-carbonyl)-benzamide,
N-[(1R,1S,2R)-1-Benzyl-2-hydroxy-3-(3-trifluoromethoxy-benzylamino)-propyl]-3-(methanesulfonyl-methyl-amino)-5-(2-thiophen-2-yl-pyrrolidine-1-carbonyl)-benzamide,
N-[(1R,1S,2R)-1-Benzyl-2-hydroxy-3-(3-trifluoromethoxy-benzylamino)-propyl]-3-methyl-5-(2-thiophen-2-yl-pyrrolidine-1-carbonyl)-benzamide, and
N-[(1S,2R)-1-Benzyl-2-hydroxy-3-(3-trifluoromethyl-benzylamino)-propyl]-3-(2-cyclopentyl-pyrrolidine-1-carbonyl)-benzamide,
   or pharmaceutically acceptable salts thereof.

One embodiment of the invention is a process for preparing a compound of formula Ia as defined in the embodiments, which process comprises reacting a compound of formula VI with a compound of formula IV

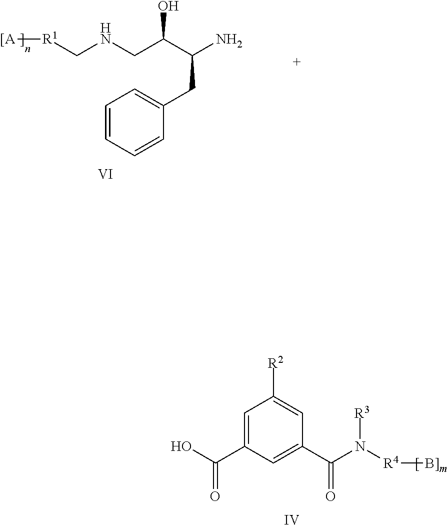

wherein A, B, n, m, R¹, R², R³ are as defined in the embodiments.

One embodiment of the invention is a compound of formula Ia for use as therapeutically active substance.

One embodiment of the invention is a compound of formula I for use as therapeutically active substance.

One embodiment of the invention is a compound of formula Ia for the use as inhibitor of BACE2 activity.

One embodiment of the invention is a compound of formula I for the use as inhibitor of BACE2 activity.

One embodiment of the invention is the use of a compound of formula I as therapeutically active substance for the therapeutic and/or prophylactic treatment of diabetes, particularly type 2 diabetes.

One embodiment of the invention is a compound of formula Ia for the use as therapeutically active substance for the therapeutic and/or prophylactic treatment of diabetes, particularly type 2 diabetes.

One embodiment of the invention is a compound of formula Ia for the use as therapeutically active substance for the therapeutic and/or prophylactic treatment of type 2 diabetes.

One embodiment of the invention is a pharmaceutical composition comprising a compound of formula Ia and a pharmaceutically acceptable carrier and/or a pharmaceutically acceptable auxiliary substance.

One embodiment of the invention is the use of a compound of formula Ia for the manufacture of a medicament for the therapeutic and/or prophylactic treatment of diabetes, particularly type 2 diabetes.

One embodiment of the invention is the use of a compound of formula I for the manufacture of a medicament for the therapeutic and/or prophylactic treatment of diabetes, particularly type 2 diabetes.

One embodiment of the invention is the use of a compound of formula I for the manufacture of a medicament for the therapeutic and/or prophylactic treatment of type 2 diabetes.

One embodiment of the invention is the use of a compound of formula Ia for the manufacture of a medicament for the therapeutic and/or prophylactic treatment of type 2 diabetes.

One embodiment of the invention is a method for the use of a compound of formula Ia in inhibition of BACE2 activity, particularly for the therapeutic and/or prophylactic treatment of diabetes or type 2 diabetes, which method comprises administering a compound of formula Ia to a human being or animal.

One embodiment of the invention is a method for the use of a compound of formula I in inhibition of BACE2 activity, particularly for the therapeutic and/or prophylactic treatment of diabetes or type 2 diabetes, which method comprises administering a compound of formula Ito a human being or animal.

One embodiment of the invention is a method for the use of a compound of formula I for the therapeutic and/or prophylactic treatment of diabetes or type 2 diabetes, which method comprises administering a compound of formula Ito a human being or animal

Furthermore, the invention includes all optical isomers, i.e. diastereoisomers, diastereomeric mixtures, racemic mixtures, all their corresponding enantiomers and/or tautomers as well as their solvates.

The compounds of formula I can contain one or more asymmetric centers and can therefore occur as racemates, racemic mixtures, single enantiomers, diastereomeric mixtures and individual diastereomers. Additional asymmetric centers can be present depending upon the nature of the various substituents on the molecule. Each such asymmetric centre will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations can be achieved as known in the art by appropriate modification of the methodology disclosed herein. Their absolute stereochemistry can be determined by the x-ray crystallography of crystalline products or crystalline intermediates which are derivatized, if necessary, with a reagent containing an asymmetric centre of known absolute configuration. If desired, racemic mixtures of the compounds can be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

Specifically, the compound of formula I can be a compound of formula Ia

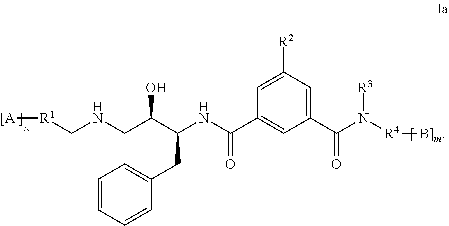

In the embodiments, where optically pure enantiomers are provided, optically pure enantiomer means that the compound contains >90% of the desired isomer by weight, preferably >95% of the desired isomer by weight, or more preferably >99% of the desired isomer by weight, said weight percent based upon the total weight of the isomer(s) of the compound. Chirally pure or chirally enriched compounds can be prepared by chirally selective synthesis or by separation of enantiomers. The separation of enantiomers can be carried out on the final product or alternatively on a suitable intermediate.

A compound of formula I can also be present in its respective tautomeric form.

The compounds of formula I can be prepared in accordance with the following schemes. The starting material is commercially available or can be prepared in accordance with known methods. Any previously defined residues and variables will continue to have the previously defined meaning unless otherwise indicated.

The preparation of compounds of formula I of the present invention may be carried out in sequential or convergent synthetic routes. Syntheses of the compounds of the invention are shown in the following scheme. The skills required for carrying out the reactions and purifications of the resulting products are known to those skilled in the art. The substituents and indices used in the following description of the processes have the significance given herein before unless indicated to the contrary. In more detail, the compounds of formula I can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). We find it convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

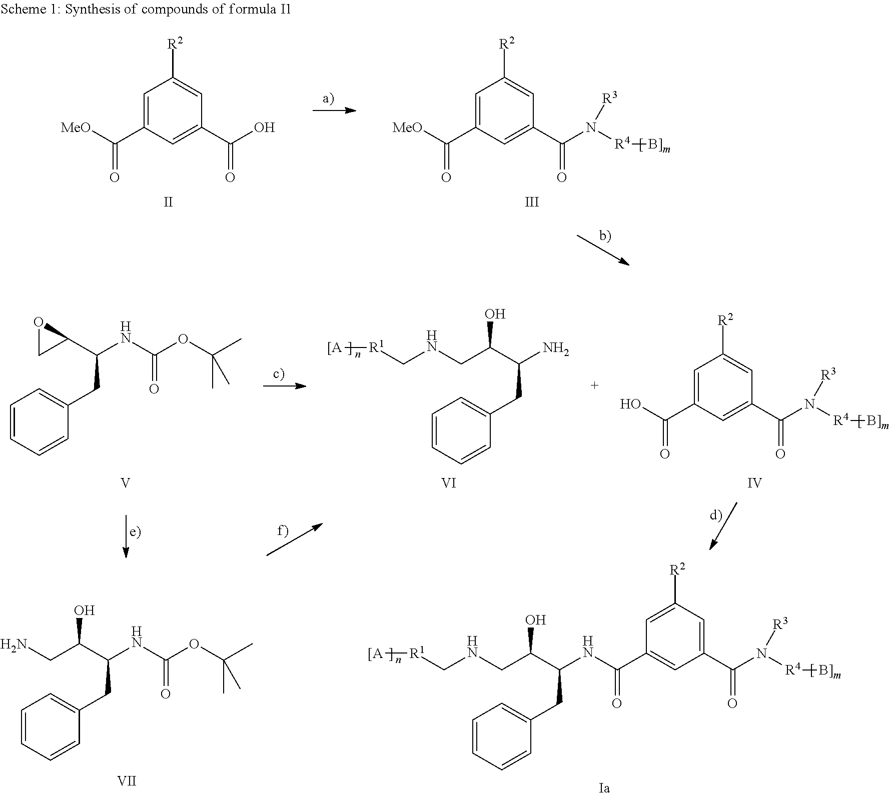

a) Aromatic acids II are either commercially available or can be synthesized according to methods known. These compounds can be coupled with suitable amines in the presence of a suitable coupling reagent (e.g. HATU, TBTU, EDCI and the like) in the presence of a suitable base (e.g. NEt₃, DIPEA and the like) to access amide derivative III.
b) The ester functionality in III can be cleaved in the presence of a suitable base (e.g. LiOH.H₂O and the like) to access acid derivatives IV.
c) Epoxide V is commercially available and can be reacted with suitable amines in the presence of a suitable base (e.g. NEt₃ and the like) to access the respective protected amino-alcohol from which the Boc-protecting group can conveniently be cleaved in the presence of a suitable acid (e.g. TFA, HCl and the like) to access the amino-alcohol VI.
d) Coupling of acid derivatives IV with amino-alcohol VI can be affected through suitable coupling reagents (e.g. HATU, TBTU, EDCI and the like) in the presence of a suitable base (e.g. NEt₃, DIPEA and the like) to access final amide derivatives I.
e) Epoxide V is commercially available and can be reacted with a suitable ammonia equivalent to access protected amino-alcohol VII.
f) The free amino-functionality in amino-alcohol VII can be reacted with suitable aldehydes under reductive conditions in the presence of a reducing agent (e.g. NaBH₄, NaBH(OAc)₃ and the like) to access the Boc-protected intermediate. The Boc-group can conveniently be cleaved in the presence of a suitable acid (e.g. TFA, HCl and the like) to access the amino-alcohol VI.

The corresponding pharmaceutically acceptable salts with acids can be obtained by standard methods known to the person skilled in the art, e.g. by dissolving the compound of formula I in a suitable solvent such as e.g. dioxane or tetrahydrofuran and adding an appropriate amount of the corresponding acid. The products can usually be isolated by filtration or by chromatography. The conversion of a compound of formula I into a pharmaceutically acceptable salt with a base can be carried out by treatment of such a compound with such a base. One possible method to form such a salt is e.g. by addition of 1/n equivalents of a basic salt such as e.g. M(OH)ₙ, wherein M=metal or ammonium cation and n=number of hydroxide anions, to a solution of the compound in a suitable solvent (e.g. ethanol, ethanol-water mixture, tetrahydrofuran-water mixture) and to remove the solvent by evaporation or lyophilisation.

Insofar as their preparation is not described in the examples, the compounds of formula I as well as all intermediate products can be prepared according to analogous methods or according to the methods set forth herewithin. Starting materials are commercially available, known in the art or can be prepared by methods known in the art or in analogy thereto.

It will be appreciated that the compounds of general formula I in this invention can be derivatised at functional groups to provide derivatives which are capable of conversion back to the parent compound in vivo.

### Pharmaceutical Compositions

The compounds of formula I and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The compounds of formula I and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also an object of the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

### EXAMPLES

The following examples illustrate the present invention without limiting it, but serve merely as representative thereof.

### Example A

Tablets of the following composition are manufactured in the usual manner:

TABLE 2
Possible tablet composition

| | mg/tablet | | | |
|---|---|---|---|---|
| ingredient | 5 | 25 | 100 | 500 |
| compound of formula I | 5 | 25 | 100 | 500 |
| lactose anhydrous DTG | 125 | 105 | 30 | 150 |
| Sta-Rx 1500 | 6 | 6 | 6 | 60 |
| microcrystalline cellulose | 30 | 30 | 30 | 45 |
| magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured:

TABLE 3
Possible capsule ingredient composition

| | mg/capsule | | | |
|---|---|---|---|---|
| ingredient | 5 | 25 | 100 | 500 |
| compound of formula I | 5 | 25 | 100 | 500 |
| hydrous lactose | 159 | 123 | 148 | — |
| corn starch | 25 | 35 | 40 | 70 |
| talk | 10 | 15 | 10 | 25 |
| magnesium stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula I, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatine capsules.

### Example B-2

Soft Gelatine Capsules of the following composition are manufactured:

TABLE 4
Possible soft gelatine capsule ingredient composition

| ingredient | mg/capsule |
|---|---|
| compound of formula I | 5 |
| yellow wax | 8 |
| hydrogenated soybean oil | 8 |
| partially hydrogenated plant oils | 34 |
| soybean oil | 110 |
| Total | 165 |

TABLE 5
Possible soft gelatine capsule composition

| ingredient | mg/capsule |
|---|---|
| gelatine | 75 |
| glycerol 85% | 32 |
| karion 83 | 8 (dry matter) |
| titanium dioxide | 0.4 |
| iron oxide yellow | 1.1 |
| Total | 116.5 |

### Manufacturing Procedure

The compound of formula I is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 6
Possible suppository composition

| ingredient | mg/supp. |
|---|---|
| compound of formula I | 15 |
| suppository mass | 1285 |
| Total | 1300 |

### Manufacturing Procedure

The suppository mass is melted in a glass or steel vessel, mixed thoroughly and cooled to 45° C. Thereupon, the finely powdered compound of formula I is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 7
Possible injection solution composition

| ingredient | mg/injection solution. |
|---|---|
| compound of formula I | 3 |
| polyethylene glycol 400 | 150 |
| acetic acid | q.s. ad pH 5.0 |
| water for injection solutions | ad 1.0 ml |

### Manufacturing Procedure

The compound of formula I is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 8
Possible sachet composition

| ingredient | mg/sachet |
|---|---|
| compound of formula I | 50 |
| lactose, fine powder | 1015 |
| microcrystalline cellulose (AVICEL PH 102) | 1400 |
| sodium carboxymethyl cellulose | 14 |
| polyvinylpyrrolidon K 30 | 10 |
| magnesium stearate | 10 |
| flavoring additives | 1 |
| Total | 2500 |

### Manufacturing Procedure

The compound of formula I is mixed with lactose, microcrystalline cellulose and sodium carboxymethyl cellulose and granulated with a mixture of polyvinylpyrrolidon in water. The granulate is mixed with magnesium stearate and the flavoring additives and filled into sachets.

### Example 1

### N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide

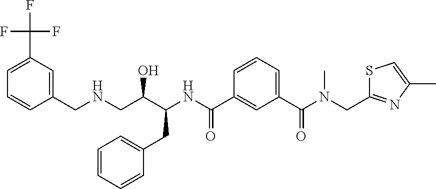

### a) N-Methyl-N-(4-methyl-thiazol-2-ylmethyl)-isophthalamic acid methyl ester

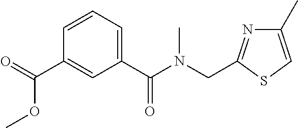

A mixture of 3-(methoxycarbonyl)benzoic acid (5.14 g, 28.5 mmol) and TBTU (11.3 g, 34.2 mmol) in DMF (30 ml) was stirred at 22° C. for 30 min. N-methyl-1-(4-methylthiazol-2-yl)methanamine (4.46 g, 31.4 mmol,) and DIPEA (5.53 g, 7.47 ml, 42.8 mmol,) were then added to the activated ester. The reaction mixture was stirred at 22° C. for 16 hr and was poured into 300 mL 1 M HCl and extracted with DCM (2×200 mL). The organic layers were combined, washed with 1 M NaHCO₃ (2×100 mL), dried over MgSO₄ and concentrated in vacuo. The product was used without further purification. MH⁺: 305.1

### b) N-Methyl-N-(4-methyl-thiazol-2-ylmethyl)-isophthalamic acid

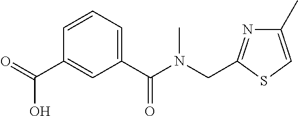

Methyl 3-(methyl((4-methylthiazol-2-yl)methyl)carbamoyl)benzoate (8.796 g, 27.5 mmol) was treated with LiOH.H₂O (4.61 g, 110 mmol) at 22° C. for 16 hr in a mixture of THF (40 mL) and water (10 mL). The crude reaction mixture was concentrated in vacuo and poured into 100 mL DCM and extracted with H₂O (2×100 mL). The aqueous layer was acidified with 4 M HCl aq. and back-extracted with DCM (2×75 mL). The organic layers were combined, washed with 1 M HCl (1×50 mL) and sat. NaCl (1×50 mL). The organic layers were dried over MgSO₄ and concentrated in vacuo to yield 6.4 g (80%) of the title compound as light-brown solid. MH⁺: 291.0.

### c) [(1S,2R)-1-Benzyl-2-hydroxy-3-(3-trifluoromethyl-benzylamino)-propyl]-carbamic acid tert-butyl ester

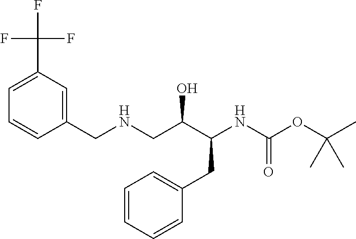

A mixture of tert-butyl (S)-1-((S)-oxiran-2-yl)-2-phenylethylcarbamate (183 mg, 0.695 mmol) and (3-(trifluoromethyl)phenyl)methanamine (305 mg, 1.74 mmol) in 2-propanol (4 mL) was refluxed for 16 hr. The reaction mixture was cooled to room temperature and all volatiles were removed under reduced pressure. The residue was poured into 20 mL 1 M KHSO₄ and extracted with DCM (2×15 mL). The organic layers were concentrated and the residue was used without further purification in the subsequent step. MH⁺: 439.3

### d) (2R,3S)-3-Amino-4-phenyl-1-(3-trifluoromethyl-benzylamino)-butan-2-ol, hydro-chloride

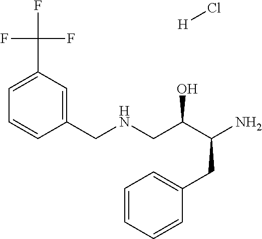

[(1S,2R)-1-Benzyl-2-hydroxy-3-(3-trifluoromethyl-b enzylamino)-propyl]-carbamic acid tert-butyl ester (0.6950 mol) was treated with HCl in dioxane (1.74 mL, 6.95 mmol) at 22° C. for 16 h. The reaction mixtures were poured into 20 mL diethyl ether. The hydrochloride salt was filtered through sintered glass, washed with diethyl ether and dried to yield 184 mg (71%) of the title compound. MH⁺: 339.2.

### e) N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide

A mixture of N-Methyl-N-(4-methyl-thiazol-2-ylmethyl)-isophthalamic acid (46 mg, 0.15 mmol) and TBTU (74.5 mg, 0.225 mmol) in DMF (4 mL) was stirred at 22° C. for 30 min. (2R,3S)-3-Amino-4-phenyl-1-(3-trifluoromethyl-benzylamino)-butan-2-ol, hydrochloride (62, 0.165 mmol) and DIPEA (58.2 mg, 78.6 μl, 0.45 mmol) were then added to the activated esters. The reaction mixture was stirred at 22° C. for 2 hr. The crude reaction mixtures were concentrated in vacuo and purified by preparative HPLC on reversed phase eluting with a gradient formed from acetonitrile, water and NEt₃ to yield after evaporation of the product containing fractions 52 mg (38%) of the title compound. MH⁺: 611.5.

### Example 2

### N-[(1S,2R)-1-Benzyl-2-hydroxy-3-(3-trifluoromethoxy-benzylamino)-propyl]-N′-methyl-N′-(4-methyl-thiazol-2-ylmethyl)-isophthalamide

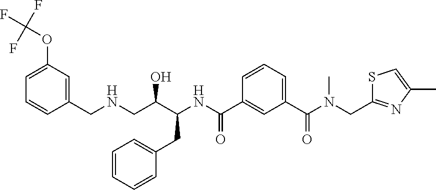

### a) [(1S,2R)-1-Benzyl-2-hydroxy-3-(3-trifluoromethoxy-benzylamino)-propyl]-carbamic acid tert-butyl ester

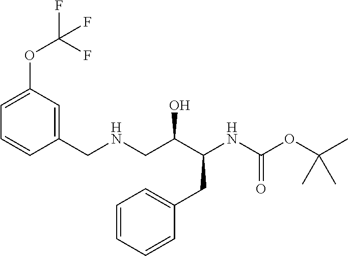

In analogy to the procedure described for the synthesis of [(1S,2R)-1-Benzyl-2-hydroxy-3-(3-trifluoromethyl-benzylamino)-propyl]-carbamic acid tert-butyl ester the title compound was prepared from tert-butyl (S)-1-((S)-oxiran-2-yl)-2-phenylethylcarbamate and (3-(trifluoromethoxy)phenyl)methanamine. MH⁺: 455.3.

### b) (2R,3S)-3-Amino-4-phenyl-1-(3-trifluoromethoxy-benzylamino)-butan-2-ol, hydro-chloride

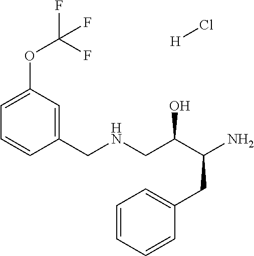

In analogy to the procedure described for the synthesis of (2R,3S)-3-Amino-4-phenyl-1-(3-trifluoromethyl-benzylamino)-butan-2-ol, hydrochloride the title compound was prepared from [(1S,2R)-1-Benzyl-2-hydroxy-3-(3-trifluoromethoxy-benzylamino)-propyl]-carbamic acid tert-butyl ester through protecting group cleavage with HCl. MH⁺: 355.2.

### c) N-[(1S,2R)-1-Benzyl-2-hydroxy-3-(3-trifluoromethoxy-benzylamino)-propyl]-N′-methyl-N′-(4-methyl-thiazol-2-ylmethyl)-isophthalamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and N-Methyl-N-(4-methyl-thiazol-2-ylmethyl)-isophthalamic acid. MH⁺: 627.5.

### Example 3

### N-[(1S,2R)-1-Benzyl-2-hydroxy-3-(3-methoxy-benzylamino)-propyl]-N′-methyl-N′-(4-methyl-thiazol-2-ylmethyl)-isophthalamide

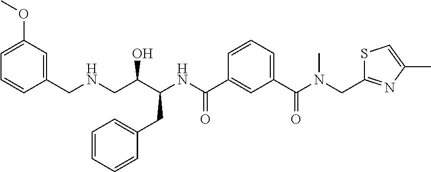

### a) [(1S,2R)-1-Benzyl-2-hydroxy-3-(3-methoxy-benzylamino)-propyl]-carbamic acid tert-butyl ester

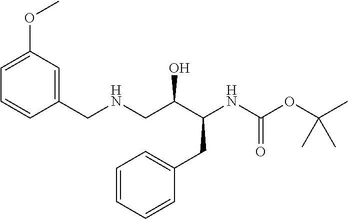

In analogy to the procedure described for the synthesis of [(1S,2R)-1-Benzyl-2-hydroxy-3-(3-trifluoromethyl-benzylamino)-propyl]-carbamic acid tert-butyl ester the title compound was prepared from tert-butyl (S)-1-((S)-oxiran-2-yl)-2-phenylethylcarbamate and (3-(methoxy)phenyl)methanamine. MH⁺: 415.3.

### b) (2R,3S)-3-Amino-1-(3-methoxy-benzylamino)-4-phenyl-butan-2-ol; hydrochloride

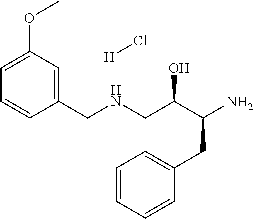

In analogy to the procedure described for the synthesis of (2R,3S)-3-Amino-4-phenyl-1-(3-trifluoromethyl-benzylamino)-butan-2-ol, hydrochloride the title compound was prepared from [(1S,2R)-1-Benzyl-2-hydroxy-3-(3-methoxy-benzylamino)-propyl]-carbamic acid tert-butyl ester through protecting group cleavage with HCl. MH⁺: 315.2.

### c) N-[(1S,2R)-1-Benzyl-2-hydroxy-3-(3-methoxy-benzylamino)-propyl]-N′-methyl-N′-(4-methyl-thiazol-2-ylmethyl)-isophthalamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-(3-methoxy-benzylamino)-4-phenyl-butan-2-ol; hydrochloride and N-Methyl-N-(4-methyl-thiazol-2-ylmethyl)-isophthalamic acid. MH⁺: 573.3.

### Example 4

### N-[(1S,2R)-1-Benzyl-2-hydroxy-3-(3-trifluoromethoxy-benzylamino)-propyl]-3-(2-pyridin-4-yl-pyrrolidine-1-carbonyl)-benzamide

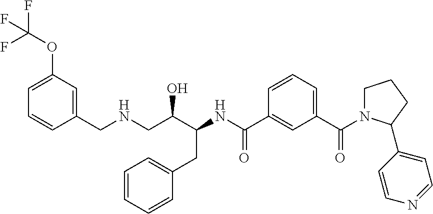

### a) 3-(2-Pyridin-4-yl-pyrrolidine-1-carbonyl)-benzoic acid methyl ester

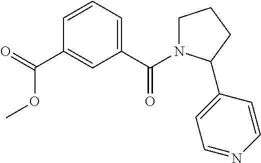

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 4-Pyrrolidin-2-yl-pyridine. MH⁺: 311.2.

### b) 3-(2-Pyridin-4-yl-pyrrolidine-1-carbonyl)-benzoic acid

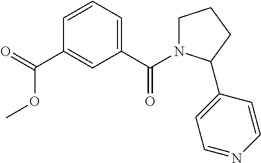

In analogy to the procedure described for the synthesis of N-Methyl-N-(4-methyl-thiazol-2-ylmethyl)-isophthalamic acid the title compound was prepared from 3-(2-Pyridin-4-yl-pyrrolidine-1-carbonyl)-benzoic acid methyl ester through cleavage of the ester. MH⁺: 297.2.

### c) N-[(1S,2R)-1-Benzyl-2-hydroxy-3-(3-trifluoromethoxy-benzylamino)-propyl]-3-(2-pyridin-4-yl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(2-Pyridin-4-yl-pyrrolidine-1-carbonyl)-benzoic acid. MH⁺: 633.5.

### Example 5

### N-[(1S,2R)-1-Benzyl-2-hydroxy-3-(3-trifluoromethyl-benzylamino)-propyl]-3-(2-pyridin-4-yl-pyrrolidine-1-carbonyl)-benzamide

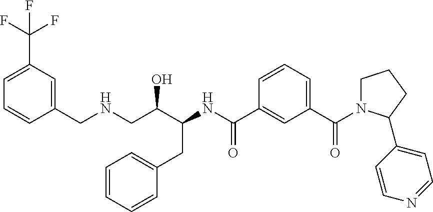

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-(2-Pyridin-4-yl-pyrrolidine-1-carbonyl)-benzoic acid. MH⁺: 617.5.

### Example 6

### N-[(1S,2R)-1-Benzyl-2-hydroxy-3-(3-trifluoromethoxy-benzylamino)-propyl]-N′-pyridin-2-ylmethyl-isophthalamide

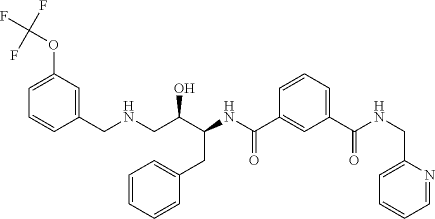

### a) N-Pyridin-2-ylmethyl-isophthalamic acid methyl ester

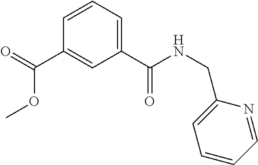

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and C-Pyridin-2-yl-methylamine. MH⁺: 271.2.

### b) N-Pyridin-2-ylmethyl-isophthalamic acid

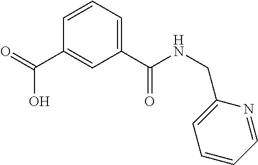

In analogy to the procedure described for the synthesis of N-Methyl-N-(4-methyl-thiazol-2-ylmethyl)-isophthalamic acid the title compound was prepared from N-Pyridin-2-ylmethyl-isophthalamic acid methyl ester through cleavage of the ester. MH⁺: 257.1

### c) N-[(1S,2R)-1-Benzyl-2-hydroxy-3-(3-trifluoromethoxy-benzylamino)-propyl]-N′-pyridin-2-ylmethyl-isophthalamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and N-Pyridin-2-ylmethyl-isophthalamic acid. MH⁺: 593.5.

### Example 7

### N-[(1S,2R)-1-Benzyl-2-hydroxy-3-(3-trifluoromethyl-benzylamino)-propyl]-N′-pyridin-2-ylmethyl-isophthalamide

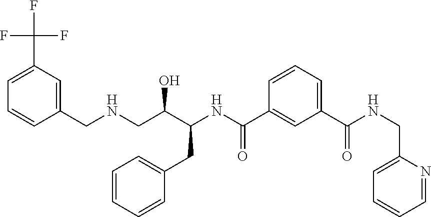

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and N-Pyridin-2-ylmethyl-isophthalamic acid. MH⁺: 577.5.

### Example 8

### N-[(1S,2R)-1-Benzyl-2-hydroxy-3-(3-trifluoromethoxy-benzylamino)-propyl]-3-((1R,5S)-1,8,8-trimethyl-3-aza-bicyclo[3.2.1]octane-3-carbonyl)-benzamide

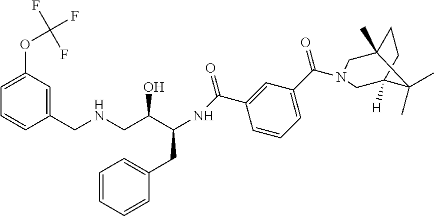

### a) 3-((1R,5S)-1,8,8-Trimethyl-3-aza-bicyclo[3.2.1]octane-3-carbonyl)-benzoic acid methyl ester

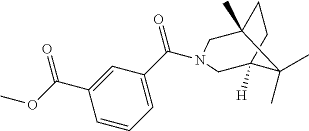

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and (1R,5S)-1,8,8-trimethyl-3-azabicyclo[3.2.1]octane hydrochloride. MH⁺: 316.2.

### b) 3-((1R,5S)-1,8,8-Trimethyl-3-aza-bicyclo[3.2.1]octane-3-carbonyl)-benzoic acid

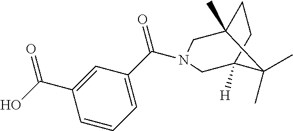

In analogy to the procedure described for the synthesis of N-Methyl-N-(4-methyl-thiazol-2-ylmethyl)-isophthalamic acid the title compound was prepared from 3-((1R,5S)-1,8,8-Trimethyl-3-aza-bicyclo[3.2.1]octane-3-carbonyl)-benzoic acid methyl ester through cleavage of the ester. MH⁺: 302.2.

### c) N-[(1S,2R)-1-Benzyl-2-hydroxy-3-(3-trifluoromethoxy-benzylamino)-propyl]-3-((1R,5S)-1,8,8-trimethyl-3-aza-bicyclo[3.2.1]octane-3-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-((1R,5S)-1,8,8-Trimethyl-3-aza-bicyclo[3.2.1]octane-3-carbonyl)-benzoic acid. MH⁺: 638.6.

### Example 9

### N-[(1S,2R)-1-Benzyl-2-hydroxy-3-(3-trifluoromethoxy-benzylamino)-propyl]-3-(3-pyridin-2-yl-pyrrolidine-1-carbonyl)-benzamide

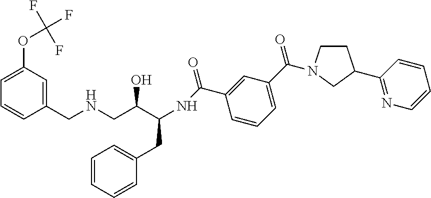

### a) 3-(3-Pyridin-2-yl-pyrrolidine-1-carbonyl)-benzoic acid methyl ester

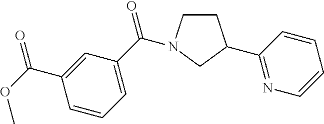

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 2-Pyrrolidin-3-yl-pyridine. MH⁺: 311.2.

### b) 3-(3-Pyridin-2-yl-pyrrolidine-1-carbonyl)-benzoic acid

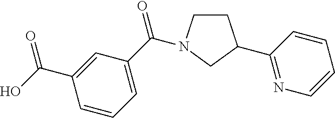

In analogy to the procedure described for the synthesis of N-Methyl-N-(4-methyl-thiazol-2-ylmethyl)-isophthalamic acid the title compound was prepared from 3-(3-Pyridin-2-yl-pyrrolidine-1-carbonyl)-benzoic acid methyl ester through cleavage of the ester. MH⁺: 297.1.

### c) N-[(1S,2R)-1-Benzyl-2-hydroxy-3-(3-trifluoromethoxy-benzylamino)-propyl]-3-(3-pyridin-2-yl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(3-Pyridin-2-yl-pyrrolidine-1-carbonyl)-benzoic acid. MH⁺: 633.6.

### Example 10

### N-[(1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide

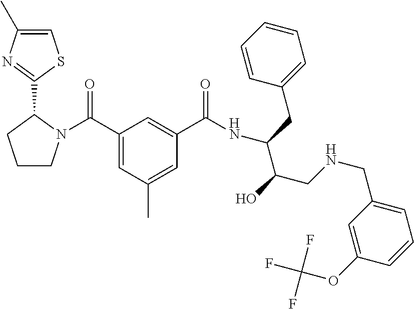

### a) 3-Methyl-5-[(R)-2-(4-methyl-thiazol-2-yl)-pyrrolidine-1-carbonyl]-benzoic acid methyl ester

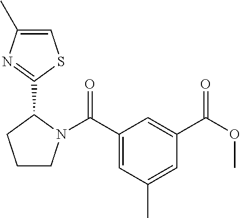

In analogy to the procedure described for the synthesis of N-Methyl-N-(4-methyl-thiazol-2-ylmethyl)-isophthalamic acid methyl ester the title compound was prepared from 5-Methyl-isophthalic acid monomethyl ester and 4-Methyl-2-(R)-pyrrolidin-2-yl-thiazole. MH⁺: 345.2.

### b) 3-Methyl-5-[(R)-2-(4-methyl-thiazol-2-yl)-pyrrolidine-1-carbonyl]-benzoic acid

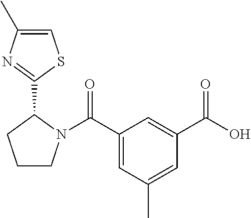

In analogy to the procedure described for the synthesis of N-Methyl-N-(4-methyl-thiazol-2-ylmethyl)-isophthalamic acid the title compound was prepared from 3-Methyl-5-[(R)-2-(4-methyl-thiazol-2-yl)-pyrrolidine-1-carbonyl]-benzoic acid methyl ester through cleavage of the ester. MH⁺: 331.2.

### c) N-[(1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-Methyl-5-[(R)-2-(4-methyl-thiazol-2-yl)-pyrrolidine-1-carbonyl]-benzoic acid. MH⁺: 667.6.

### Example 11

### N-[(1S,2R)-1-Benzyl-2-hydroxy-3-(3-trifluoromethyl-benzylamino)-propyl]-3-[(R)-2-(4-methyl-thiazol-2-yl)-pyrrolidine-1-carbonyl]-benzamide

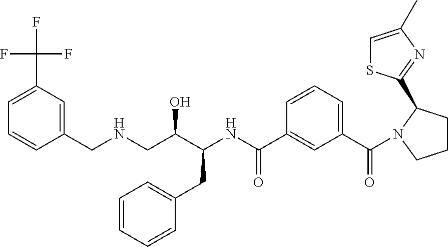

### a) 3-[(R)-2-(4-Methyl-thiazol-2-yl)-pyrrolidine-1-carbonyl]-benzoic acid methyl ester

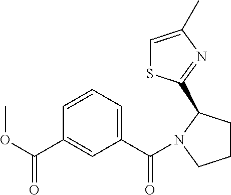

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 4-Methyl-2-(R)-pyrrolidin-2-yl-thiazole. MH⁺: 331.2.

### b) 3-[(R)-2-(4-Methyl-thiazol-2-yl)-pyrrolidine-1-carbonyl]-benzoic acid

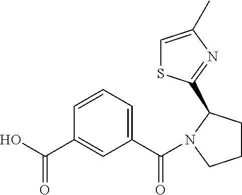

In analogy to the procedure described for the synthesis of N-Methyl-N-(4-methyl-thiazol-2-ylmethyl)-isophthalamic acid the title compound was prepared from 3-[(R)-2-(4-Methyl-thiazol-2-yl)-pyrrolidine-1-carbonyl]-benzoic acid methyl ester through cleavage of the ester. MH⁺: 317.1.

### c) N-[(1S,2R)-1-Benzyl-2-hydroxy-3-(3-trifluoromethyl-benzylamino)-propyl]-3-[(R)-2-(4-methyl-thiazol-2-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-[(R)-2-(4-Methyl-thiazol-2-yl)-pyrrolidine-1-carbonyl]-benzoic acid. MH⁺: 637.5.

### Example 12

### N-[(1S,2R)-1-Benzyl-2-hydroxy-3-(3-trifluoromethyl-benzylamino)-propyl]-3-(2-phenyl-pyrrolidine-1-carbonyl)-benzamide

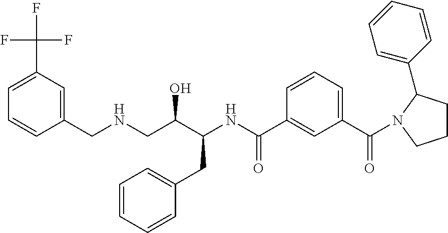

### a) 3-(2-Phenyl-pyrrolidine-1-carbonyl)-benzoic acid methyl ester

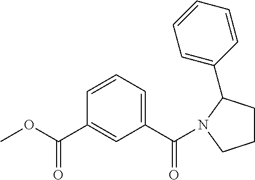

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 2-Phenyl-pyrrolidine. MH⁺: 310.2.

### b) 3-(2-Phenyl-pyrrolidine-1-carbonyl)-benzoic acid

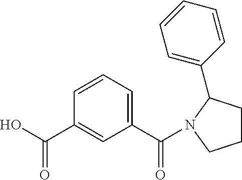

In analogy to the procedure described for the synthesis of N-Methyl-N-(4-methyl-thiazol-2-ylmethyl)-isophthalamic acid the title compound was prepared from 3-(2-Phenyl-pyrrolidine-1-carbonyl)-benzoic acid methyl ester through cleavage of the ester. MH⁺: 296.2.

### c) N-[(1S,2R)-1-Benzyl-2-hydroxy-3-(3-trifluoromethyl-benzylamino)-propyl]-3-(2-phenyl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-(2-Pyridin-4-yl-pyrrolidine-1-carbonyl)-benzoic acid. MH⁺: 616.6

### Example 13

### N-[(1S,2R)-1-Benzyl-2-hydroxy-3-(3-trifluoromethyl-benzylamino)-propyl]-3-(2-pyridin-3-yl-pyrrolidine-1-carbonyl)-benzamide

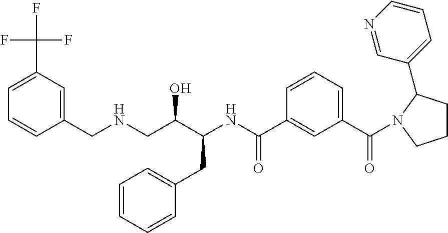

### a) 3-(2-Pyridin-3-yl-pyrrolidine-1-carbonyl)-benzoic acid methyl ester

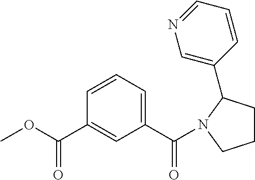

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 3-Pyrrolidin-2-yl-pyridine. MH⁺: 311.2.

### b) 3-(2-Pyridin-3-yl-pyrrolidine-1-carbonyl)-benzoic acid

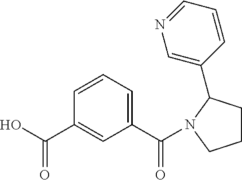

In analogy to the procedure described for the synthesis of N-Methyl-N-(4-methyl-thiazol-2-ylmethyl)-isophthalamic acid the title compound was prepared from 3-(2-Pyridin-3-yl-pyrrolidine-1-carbonyl)-benzoic acid methyl ester through cleavage of the ester. MH⁺: 297.2.

### c) N-[(1S,2R)-1-Benzyl-2-hydroxy-3-(3-trifluoromethyl-benzylamino)-propyl]-3-(2-pyridin-3-yl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-(2-Pyridin-3-yl-pyrrolidine-1-carbonyl)-benzoic acid. MH⁺: 617.5.

### Example 14

### N-[(1S,2R)-1-Benzyl-2-hydroxy-3-(3-trifluoromethyl-benzylamino)-propyl]-3-[2-(1-methyl-1H-pyrazol-3-yl)-pyrrolidine-1-carbonyl]-benzamide

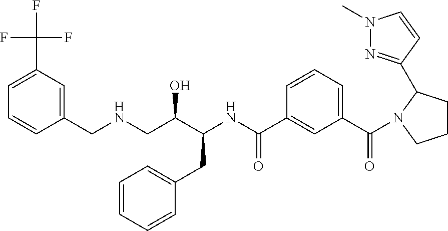

### a) 3-[2-(1-Methyl-1H-pyrazol-3-yl)-pyrrolidine-1-carbonyl]-benzoic acid methyl ester

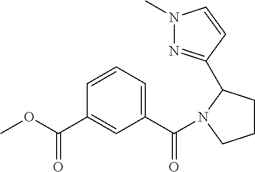

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 1-Methyl-3-pyrrolidin-2-yl-1H-pyrazole. MH⁺: 314.5.

### b) 3-[2-(1-Methyl-1H-pyrazol-3-yl)-pyrrolidine-1-carbonyl]-benzoic acid

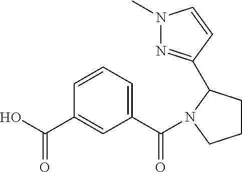

In analogy to the procedure described for the synthesis of N-Methyl-N-(4-methyl-thiazol-2-ylmethyl)-isophthalamic acid the title compound was prepared from 3-[2-(1-Methyl-1H-pyrazol-3-yl)-pyrrolidine-1-carbonyl]-benzoic acid methyl ester through cleavage of the ester. MH⁺: 300.2.

### c) N-[(1S,2R)-1-Benzyl-2-hydroxy-3-(3-trifluoromethyl-benzylamino)-propyl]-3-[2-(1-methyl-1H-pyrazol-3-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-[2-(1-Methyl-1H-pyrazol-3-yl)-pyrrolidine-1-carbonyl]-benzoic acid. MH⁺: 620.6

### Example 15

### N-[(1S,2R)-1-Benzyl-2-hydroxy-3-(3-trifluoromethyl-benzylamino)-propyl]-3-[2-(2-methyl-2H-pyrazol-3-yl)-pyrrolidine-1-carbonyl]-benzamide

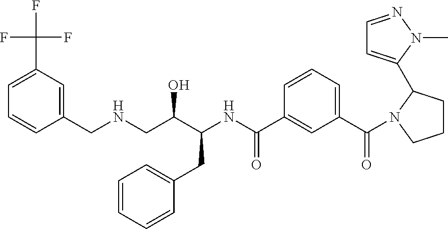

### a) 3-[2-(2-Methyl-2H-pyrazol-3-yl)-pyrrolidine-1-carbonyl]-benzoic acid methyl ester

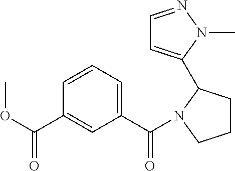

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 1-Methyl-5-pyrrolidin-2-yl-1H-pyrazole. MH⁺: 314.2.

### b) 3-[2-(2-Methyl-2H-pyrazol-3-yl)-pyrrolidine-1-carbonyl]-benzoic acid

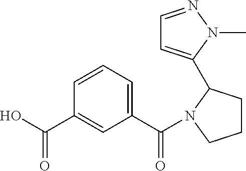

In analogy to the procedure described for the synthesis of N-Methyl-N-(4-methyl-thiazol-2-ylmethyl)-isophthalamic acid the title compound was prepared from 3-[2-(2-Methyl-2H-pyrazol-3-yl)-pyrrolidine-1-carbonyl]-benzoic acid methyl ester through cleavage of the ester. MH⁺: 300.2.

### c) N-[(1S,2R)-1-Benzyl-2-hydroxy-3-(3-trifluoromethyl-benzylamino)-propyl]-3-[2-(2-methyl-2H-pyrazol-3-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-[2-(2-Methyl-2H-pyrazol-3-yl)-pyrrolidine-1-carbonyl]-benzoic acid. MH⁺: 620.6

### Example 16

### N-[(1S,2R)-1-Benzyl-2-hydroxy-3-(3-trifluoromethyl-benzylamino)-propyl]-3-[2-(3-methyl-isoxazol-5-yl)-pyrrolidine-1-carbonyl]-benzamide

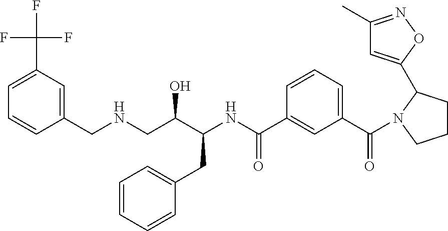

### a) 3-[2-(3-Methyl-isoxazol-5-yl)-pyrrolidine-1-carbonyl]-benzoic acid methyl ester

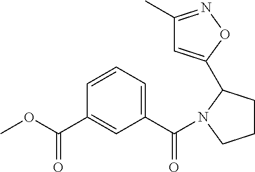

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 3-Methyl-5-pyrrolidin-2-yl-isoxazole. MH⁺: 315.1.

### b) 3-[2-(3-Methyl-isoxazol-5-yl)-pyrrolidine-1-carbonyl]-benzoic acid

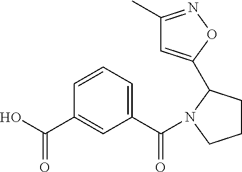

In analogy to the procedure described for the synthesis of N-Methyl-N-(4-methyl-thiazol-2-ylmethyl)-isophthalamic acid the title compound was prepared from 3-[2-(3-Methyl-isoxazol-5-yl)-pyrrolidine-1-carbonyl]-benzoic acid methyl ester through cleavage of the ester. MH⁺: 301.2.

### c) N-[(1S,2R)-1-Benzyl-2-hydroxy-3-(3-trifluoromethyl-benzylamino)-propyl]-3-[2-(3-methyl-isoxazol-5-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-[2-(3-Methyl-isoxazol-5-yl)-pyrrolidine-1-carbonyl]-benzoic acid. MH⁺: 621.5.

### Example 17

### N-[(1S,2R)-1-Benzyl-2-hydroxy-3-(3-trifluoromethyl-benzylamino)-propyl]-3-[2-(3-methyl-[1,2,4]oxadiazol-5-yl)-pyrrolidine-1-carbonyl]-benzamide

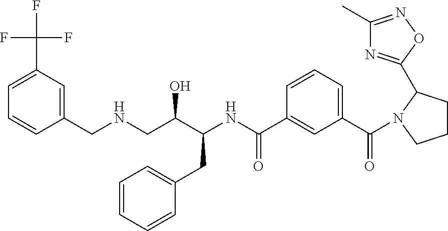

### a) 3-[2-(3-Methyl-[1,2,4]oxadiazol-5-yl)-pyrrolidine-1-carbonyl]-benzoic acid methyl ester

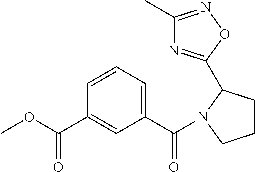

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 3-Methyl-5-pyrrolidin-2-yl-[1,2,4]oxadiazole. MH⁺: 316.1.

### b) 3-[2-(3-Methyl-[1,2,4]oxadiazol-5-yl)-pyrrolidine-1-carbonyl]-benzoic acid

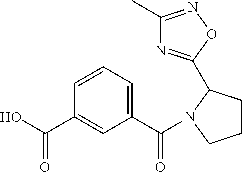

In analogy to the procedure described for the synthesis of N-Methyl-N-(4-methyl-thiazol-2-ylmethyl)-isophthalamic acid the title compound was prepared from 3-[2-(3-Methyl-[1,2,4]oxadiazol-5-yl)-pyrrolidine-1-carbonyl]-benzoic acid methyl ester through cleavage of the ester. MH⁺: 302.2.

### c) N-[(1S,2R)-1-Benzyl-2-hydroxy-3-(3-trifluoromethyl-benzylamino)-propyl]-3-[2-(3-methyl-[1,2,4]oxadiazol-5-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-[2-(3-Methyl-[1,2,4]oxadiazol-5-yl)-pyrrolidine-1-carbonyl]-benzoic acid. MH⁺: 622.5.

### Example 18

### N-[(1S,2R)-1-Benzyl-2-hydroxy-3-(3-trifluoromethyl-benzylamino)-propyl]-N′-methyl-N′-pyridin-2-ylmethyl-isophthalamide

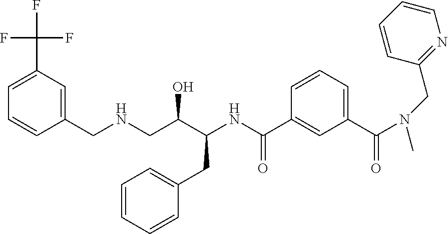

### a) N-Methyl-N-pyridin-2-ylmethyl-isophthalamic acid methyl ester

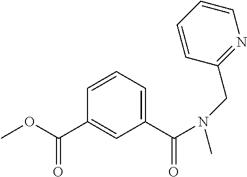

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and Methyl-pyridin-2-ylmethyl-amine. MH⁺: 285.1.

### b) N-Methyl-N-pyridin-2-ylmethyl-isophthalamic acid

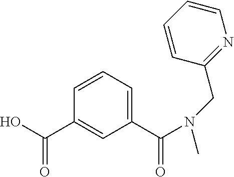

In analogy to the procedure described for the synthesis of N-Methyl-N-(4-methyl-thiazol-2-ylmethyl)-isophthalamic acid the title compound was prepared from N-Methyl-N-pyridin-2-ylmethyl-isophthalamic acid methyl ester through cleavage of the ester. MH⁺: 271.1.

### c) N-[(1S,2R)-1-Benzyl-2-hydroxy-3-(3-trifluoromethyl-benzylamino)-propyl]-N′-methyl-N′-pyridin-2-ylmethyl-isophthalamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and N-Methyl-N-pyridin-2-ylmethyl-isophthalamic acid. MH⁺: 591.5.

### Example 19

### N-[(1S,2R)-1-Benzyl-2-hydroxy-3-(3-trifluoromethyl-benzylamino)-propyl]-3-(2-thiophen-2-yl-pyrrolidine-1-carbonyl)-benzamide

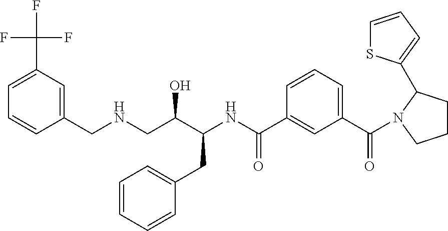

### a) 3-(2-Thiophen-2-yl-pyrrolidine-1-carbonyl)-benzoic acid methyl ester

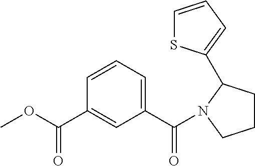

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 2-Thiophen-2-yl-pyrrolidine. MH⁺: 316.1.

### b) 3-(2-Thiophen-2-yl-pyrrolidine-1-carbonyl)-benzoic acid

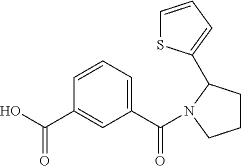

In analogy to the procedure described for the synthesis of N-Methyl-N-(4-methyl-thiazol-2-ylmethyl)-isophthalamic acid the title compound was prepared from 3-(2-Thiophen-2-yl-pyrrolidine-1-carbonyl)-benzoic acid methyl ester through cleavage of the ester. MH⁺: 302.1.

### c) N-[(1S,2R)-1-Benzyl-2-hydroxy-3-(3-trifluoromethyl-benzylamino)-propyl]-3-(2-thiophen-2-yl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-(2-Thiophen-2-yl-pyrrolidine-1-carbonyl)-benzoic acid. MH⁺: 622.5.

### Example 20

### N-[(1S,2R)-1-Benzyl-2-hydroxy-3-(3-methoxy-benzylamino)-propyl]-3-[(R)-2-(4-methyl-thiazol-2-yl)-pyrrolidine-1-carbonyl]-benzamide

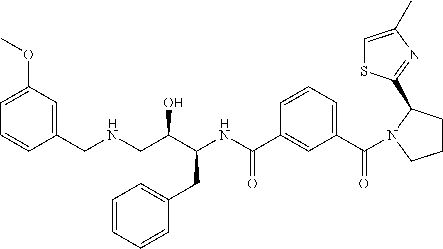

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-(3-methoxy-benzylamino)-4-phenyl-butan-2-ol; hydrochloride and 3-[(R)-2-(4-Methyl-thiazol-2-yl)-pyrrolidine-1-carbonyl]-benzoic acid. MH⁺: 599.6.

### Example 21

### N-[(1S,2R)-1-Benzyl-2-hydroxy-3-(3-trifluoromethoxy-benzylamino)-propyl]-3-[(R)-2-(4-methyl-thiazol-2-yl)-pyrrolidine-1-carbonyl]-benzamide

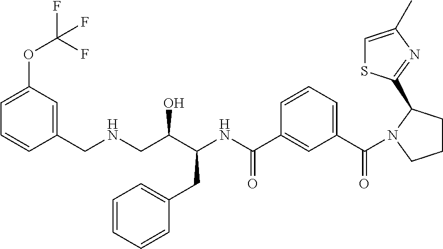

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-[(R)-2-(4-Methyl-thiazol-2-yl)-pyrrolidine-1-carbonyl]-benzoic acid. MH⁺: 653.6.

### Example 22

### N-[(1S,2R)-1-Benzyl-2-hydroxy-3-(3-methoxy-benzylamino)-propyl]-3-(2-phenyl-pyrrolidine-1-carbonyl)-benzamide

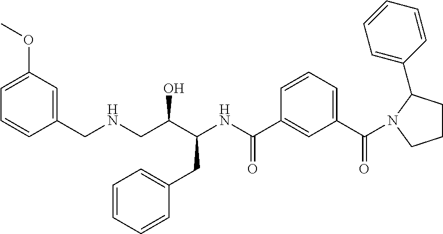

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-(3-methoxy-benzylamino)-4-phenyl-butan-2-ol; hydrochloride and 3-(2-Phenyl-pyrrolidine-1-carbonyl)-benzoic acid. MH⁺: 578.6.

### Example 23

### N-{(1S,2R)-1-Benzyl-3-[(1-ethyl-1H-pyrazol-4-ylmethyl)-amino]-2-hydroxy-propyl}-3-(2-phenyl-pyrrolidine-1-carbonyl)-benzamide

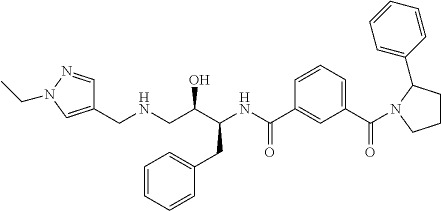

### a) {(1S,2R)-1-Benzyl-3-[(1-ethyl-1H-pyrazol-4-ylmethyl)-amino]-2-hydroxy-propyl}-carbamic acid tert-butyl ester

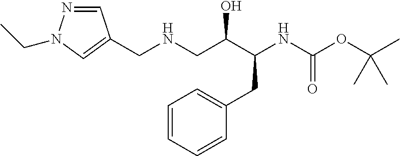

In analogy to the procedure described for the synthesis of [(1S,2R)-1-Benzyl-2-hydroxy-3-(3-trifluoromethyl-benzylamino)-propyl]-carbamic acid tert-butyl ester the title compound was prepared from tert-butyl (S)-1-((S)-oxiran-2-yl)-2-phenylethylcarbamate and C-(1-Ethyl-1H-pyrazol-4-yl)-methylamine. MH⁺: 389.4.

### b) (2R,3S)-3-Amino-1-[(1-ethyl-1H-pyrazol-4-ylmethyl)-amino]-4-phenyl-butan-2-ol; hydrochloride

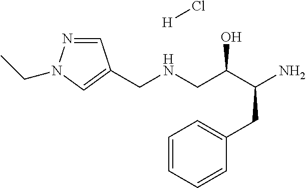

In analogy to the procedure described for the synthesis of (2R,3S)-3-Amino-4-phenyl-1-(3-trifluoromethyl-benzylamino)-butan-2-ol, hydrochloride the title compound was prepared from {(1S,2R)-1-Benzyl-3-[(1-ethyl-1H-pyrazol-4-ylmethyl)-amino]-2-hydroxy-propyl}-carbamic acid tert-butyl ester through protecting group cleavage with HCl. MH⁺: 289.3.

### c) N-{(1S,2R)-1-Benzyl-3-[(1-ethyl-1H-pyrazol-4-ylmethyl)-amino]-2-hydroxy-propyl}-3-(2-phenyl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-(2-Phenyl-pyrrolidine-1-carbonyl)-benzoic acid. MH⁺: 566.6.

### Example 24

### N-[(1S,2R)-1-Benzyl-2-hydroxy-3-(3-trifluoromethoxy-benzylamino)-propyl]-3-(2-phenyl-pyrrolidine-1-carbonyl)-benzamide

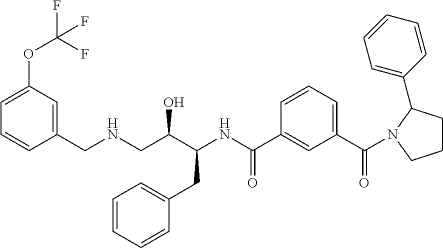

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(2-Phenyl-pyrrolidine-1-carbonyl)-benzoic acid. MH⁺: 632.6.

### Example 25

### N-[(1S,2R)-1-Benzyl-2-hydroxy-3-(3-trifluoromethoxy-benzylamino)-propyl]-3-(2-pyridin-3-yl-pyrrolidine-1-carbonyl)-benzamide

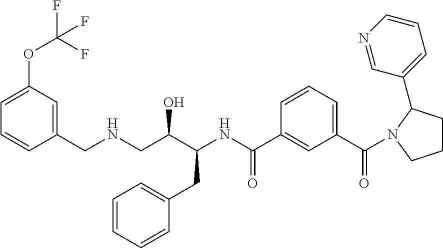

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(2-Pyridin-3-yl-pyrrolidine-1-carbonyl)-benzoic acid. MH⁺: 633.6.

### Example 26

### N-[(1S,2R)-1-Benzyl-2-hydroxy-3-(3-trifluoromethoxy-benzylamino)-propyl]-3-[2-(3-methyl-isoxazol-5-yl)-pyrrolidine-1-carbonyl]-benzamide

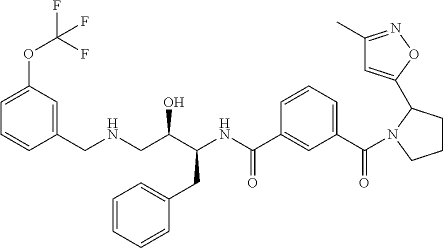

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-[2-(3-Methyl-isoxazol-5-yl)-pyrrolidine-1-carbonyl]-benzoic acid. MH⁺: 637.6.

### Example 27

### N-[(1S,2R)-1-Benzyl-2-hydroxy-3-(3-methoxy-benzylamino)-propyl]-3-[2-(5-methyl-thiophen-2-yl)-pyrrolidine-1-carbonyl]-benzamide

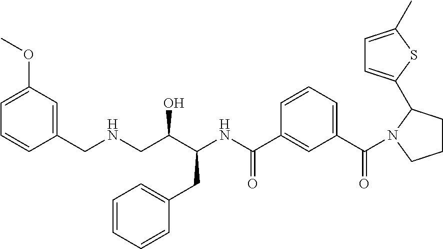

### a) 3-[2-(5-Methyl-thiophen-2-yl)-pyrrolidine-1-carbonyl]-benzoic acid methyl ester

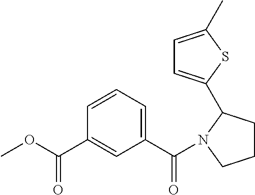

In analogy to the procedure described for the synthesis of N-Methyl-N-(4-methyl-thiazol-2-ylmethyl)-isophthalamic acid methyl ester the title compound was prepared from 3-(methoxycarbonyl)benzoic acid and 2-(5-Methyl-thiophen-2-yl)-pyrrolidine. MH⁺: 330.1.

### b) 3-[2-(5-Methyl-thiophen-2-yl)-pyrrolidine-1-carbonyl]-benzoic acid

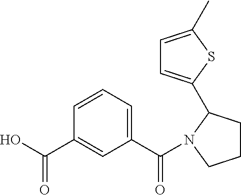

In analogy to the procedure described for the synthesis of N-Methyl-N-(4-methyl-thiazol-2-ylmethyl)-isophthalamic acid the title compound was prepared from 3-[2-(5-Methyl-thiophen-2-yl)-pyrrolidine-1-carbonyl]-benzoic acid methyl ester through cleavage of the ester. MH⁺: 316.1.

### c) N-[(1S,2R)-1-Benzyl-2-hydroxy-3-(3-methoxy-benzylamino)-propyl]-3-[2-(5-methyl-thiophen-2-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-(3-methoxy-benzylamino)-4-phenyl-butan-2-ol; hydrochloride and 3-[2-(5-Methyl-thiophen-2-yl)-pyrrolidine-1-carbonyl]-benzoic acid. MH⁺: 598.6.

### Example 28

### N-[(1S,2R)-1-Benzyl-2-hydroxy-3-(3-methoxy-benzylamino)-propyl]-N′-methyl-N′-pyridin-2-ylmethyl-isophthalamide

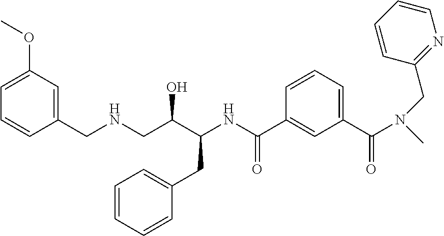

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-(3-methoxy-benzylamino)-4-phenyl-butan-2-ol; hydrochloride and N-Methyl-N-pyridin-2-ylmethyl-isophthalamic acid. MH⁺: 553.6.

### Example 29

### N-[(1S,2R)-1-Benzyl-2-hydroxy-3-(3-trifluoromethoxy-benzylamino)-propyl]-N′-methyl-N′-pyridin-2-ylmethyl-isophthalamide

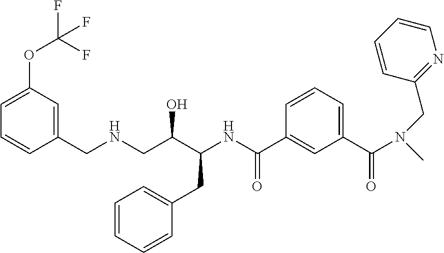

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and N-Methyl-N-pyridin-2-ylmethyl-isophthalamic acid. MH⁺: 607.6.

### Example 30

### N-[(1S,2R)-1-Benzyl-2-hydroxy-3-(3-methoxy-benzylamino)-propyl]-3-(2-thiophen-2-yl-pyrrolidine-1-carbonyl)-benzamide

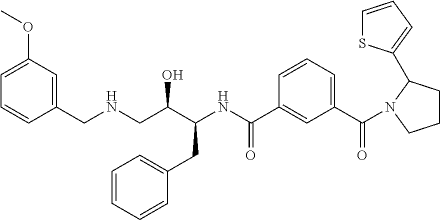

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-(3-methoxy-benzylamino)-4-phenyl-butan-2-ol; hydrochloride and 3-(2-Thiophen-2-yl-pyrrolidine-1-carbonyl)-benzoic acid. MH⁺: 584.6.

### Example 31

### N-{(1S,2R)-1-Benzyl-3-[(1-ethyl-1H-pyrazol-4-ylmethyl)-amino]-2-hydroxy-propyl}-3-(2-thiophen-2-yl-pyrrolidine-1-carbonyl)-benzamide

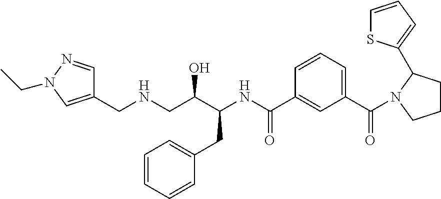

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-(2-Thiophen-2-yl-pyrrolidine-1-carbonyl)-benzoic acid. MH⁺: 572.6.

### Example 32

### N-[(1S,2R)-1-Benzyl-2-hydroxy-3-(3-trifluoromethoxy-benzylamino)-propyl]-3-(2-thiophen-2-yl-pyrrolidine-1-carbonyl)-benzamide

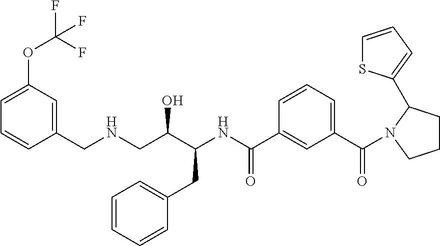

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(2-Thiophen-2-yl-pyrrolidine-1-carbonyl)-benzoic acid. MH⁺: 638.6.

### Example 33

### N-{(1S,2R)-1-Benzyl-3-[(1-ethyl-1H-pyrazol-4-ylmethyl)-amino]-2-hydroxy-propyl}-3-(methanesulfonyl-methyl-amino)-5-[(R)-2-(4-methyl-thiazol-2-yl)-pyrrolidine-1-carbonyl]-benzamide

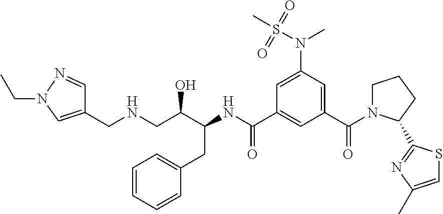

### a) 3-(Methanesulfonyl-methyl-amino)-5-[(R)-2-(4-methyl-thiazol-2-yl)-pyrrolidine-1-carbonyl]-benzoic acid methyl ester

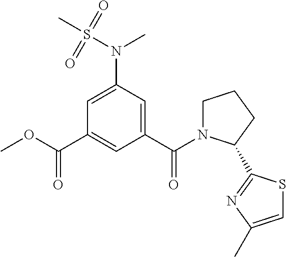

In analogy to the procedure described for the synthesis of N-Methyl-N-(4-methyl-thiazol-2-ylmethyl)-isophthalamic acid methyl ester the title compound was prepared from 5-(Methanesulfonyl-methyl-amino)-isophthalic acid monomethyl ester and 4-Methyl-2-(R)-pyrrolidin-2-yl-thiazole. MH⁺: 437.7.

### b) 3-(Methanesulfonyl-methyl-amino)-5-[(R)-2-(4-methyl-thiazol-2-yl)-pyrrolidine-1-carbonyl]-benzoic acid

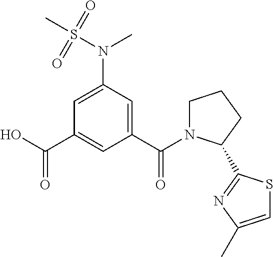

In analogy to the procedure described for the synthesis of N-Methyl-N-(4-methyl-thiazol-2-ylmethyl)-isophthalamic acid the title compound was prepared from 3-(Methanesulfonyl-methyl-amino)-5-[(R)-2-(4-methyl-thiazol-2-yl)-pyrrolidine-1-carbonyl]-benzoic acid methyl ester through cleavage of the ester. MH⁺: 424.1.

### c) N-{(1S,2R)-1-Benzyl-3-[(1-ethyl-1H-pyrazol-4-ylmethyl)-amino]-2-hydroxy-propyl}-3-(methanesulfonyl-methyl-amino)-5-[(R)-2-(4-methyl-thiazol-2-yl)-pyrrolidine-1-carbonyl]-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-(Methanesulfonyl-methyl-amino)-5-[(R)-2-(4-methyl-thiazol-2-yl)-pyrrolidine-1-carbonyl]-benzoic acid. MH⁺: 694.7.

### Example 34

### N-{(1S,2R)-1-Benzyl-3-[(1-ethyl-1H-pyrazol-4-ylmethyl)-amino]-2-hydroxy-propyl}-3-methyl-5-[(R)-2-(4-methyl-thiazol-2-yl)-pyrrolidine-1-carbonyl]-benzamide

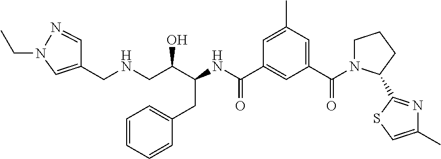

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-[(R)-2-(4-Methyl-thiazol-2-yl)-pyrrolidine-1-carbonyl]-benzoic acid. MH⁺: 601.6.

### Example 35

### N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-phenyl-pyrrolidine-1-carbonyl)-benzamide

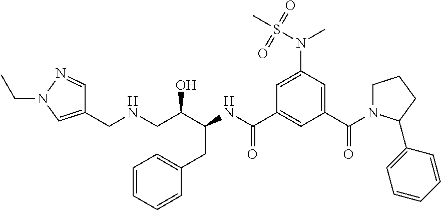

### a) 3-(Methanesulfonyl-methyl-amino)-5-(2-phenyl-pyrrolidine-1-carbonyl)-benzoic acid methyl ester

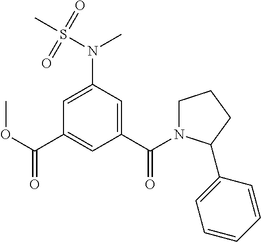

In analogy to the procedure described for the synthesis of N-Methyl-N-(4-methyl-thiazol-2-ylmethyl)-isophthalamic acid methyl ester the title compound was prepared from 5-(Methanesulfonyl-methyl-amino)-isophthalic acid monomethyl ester and 2-Phenyl-pyrrolidine. MH⁺: 417.1.

### b) 3-(Methanesulfonyl-methyl-amino)-5-(2-phenyl-pyrrolidine-1-carbonyl)-benzoic acid

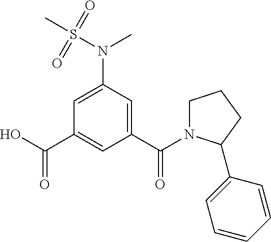

In analogy to the procedure described for the synthesis of N-Methyl-N-(4-methyl-thiazol-2-ylmethyl)-isophthalamic acid the title compound was prepared from 3-(Methanesulfonyl-methyl-amino)-5-(2-phenyl-pyrrolidine-1-carbonyl)-benzoic acid methyl ester through cleavage of the ester. MH⁺: 403.0.

### c) N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-phenyl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-(Methanesulfonyl-methyl-amino)-5-(2-phenyl-pyrrolidine-1-carbonyl)-benzoic acid. MH⁺: 673.7.

### Example 36

### N-{(1S,2R)-1-Benzyl-3-[(1-ethyl-1H-pyrazol-4-ylmethyl)-amino]-2-hydroxy-propyl}-3-methyl-5-(2-phenyl-pyrrolidine-1-carbonyl)-benzamide

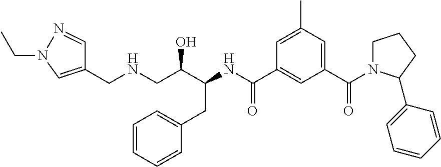

### a) 3-Methyl-5-(2-phenyl-pyrrolidine-1-carbonyl)-benzoic acid methyl ester

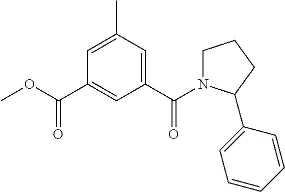

In analogy to the procedure described for the synthesis of N-Methyl-N-(4-methyl-thiazol-2-ylmethyl)-isophthalamic acid methyl ester the title compound was prepared from 5-Methyl-isophthalic acid monomethyl ester and 2-Phenyl-pyrrolidine. MH⁺: 324.1.

### b) 3-Methyl-5-(2-phenyl-pyrrolidine-1-carbonyl)-benzoic acid

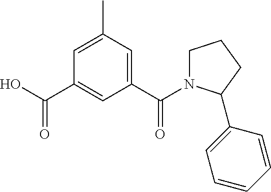

In analogy to the procedure described for the synthesis of N-Methyl-N-(4-methyl-thiazol-2-ylmethyl)-isophthalamic acid the title compound was prepared from 3-Methyl-5-(2-phenyl-pyrrolidine-1-carbonyl)-benzoic acid methyl ester through cleavage of the ester. MH⁺: 310.1.

### c) N-{(1S,2R)-1-Benzyl-3-[(1-ethyl-1H-pyrazol-4-ylmethyl)-amino]-2-hydroxy-propyl}-3-methyl-5-(2-phenyl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-Methyl-5-(2-phenyl-pyrrolidine-1-carbonyl)-benzoic acid. MH⁺: 580.7.

### Example 37

### N-{(1S,2R)-1-Benzyl-3-[(1-ethyl-1H-pyrazol-4-ylmethyl)-amino]-2-hydroxy-propyl}-5,N′-dimethyl-N′-pyridin-2-ylmethyl-isophthalamide

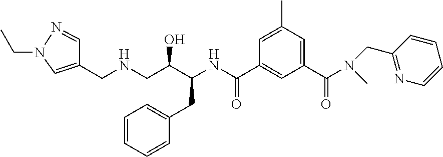

### a) 5,N-Dimethyl-N-pyridin-2-ylmethyl-isophthalamic acid methyl ester

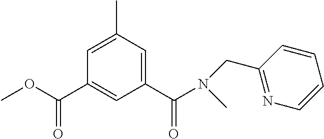

In analogy to the procedure described for the synthesis of N-Methyl-N-(4-methyl-thiazol-2-ylmethyl)-isophthalamic acid methyl ester the title compound was prepared from 5-Methyl-isophthalic acid monomethyl ester and Methyl-pyridin-2-ylmethyl-amine. MH⁺: 299.2.

### b) 5,N-Dimethyl-N-pyridin-2-ylmethyl-isophthalamic acid

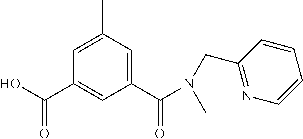

In analogy to the procedure described for the synthesis of N-Methyl-N-(4-methyl-thiazol-2-ylmethyl)-isophthalamic acid the title compound was prepared from 5,N-Dimethyl-N-pyridin-2-ylmethyl-isophthalamic acid methyl ester through cleavage of the ester. MH⁺: 285.0.

### c) N-{(1S,2R)-1-Benzyl-3-[(1-ethyl-1H-pyrazol-4-ylmethyl)-amino]-2-hydroxy-propyl}-5,N′-dimethyl-N′-pyridin-2-ylmethyl-isophthalamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 5,N-Dimethyl-N-pyridin-2-ylmethyl-isophthalamic acid. MH⁺: 555.6.

### Example 38

### N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide

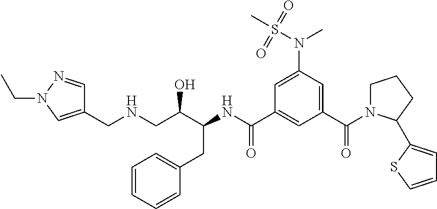

### a) 3-(Methanesulfonyl-methyl-amino)-5-(2-thiophen-2-yl-pyrrolidine-1-carbonyl)-benzoic acid methyl ester

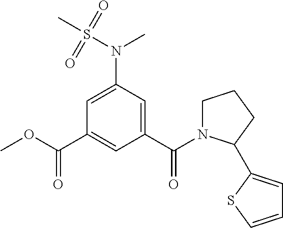

In analogy to the procedure described for the synthesis of N-Methyl-N-(4-methyl-thiazol-2-ylmethyl)-isophthalamic acid methyl ester the title compound was prepared from 5-(Methanesulfonyl-methyl-amino)-isophthalic acid monomethyl ester and 2-Thiophen-2-yl-pyrrolidine. MH⁺: 422.5.

### b) 3-(Methanesulfonyl-methyl-amino)-5-(2-thiophen-2-yl-pyrrolidine-1-carbonyl)-benzoic acid

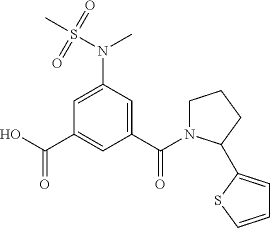

In analogy to the procedure described for the synthesis of N-Methyl-N-(4-methyl-thiazol-2-ylmethyl)-isophthalamic acid the title compound was prepared from 3-(Methanesulfonyl-methyl-amino)-5-(2-thiophen-2-yl-pyrrolidine-1-carbonyl)-benzoic acid methyl ester through cleavage of the ester. MH⁺: 409.2.

### c) N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-(Methanesulfonyl-methyl-amino)-5-(2-thiophen-2-yl-pyrrolidine-1-carbonyl)-benzoic acid. MH⁺: 679.7.

### Example 39

### N-{(1S,2R)-1-Benzyl-3-[(1-ethyl-1H-pyrazol-4-ylmethyl)-amino]-2-hydroxy-propyl}-3-methyl-5-(2-thiophen-2-yl-pyrrolidine-1-carbonyl)-benzamide

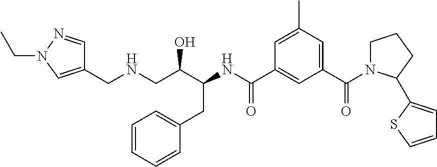

### a) 3-Methyl-5-(2-thiophen-2-yl-pyrrolidine-1-carbonyl)-benzoic acid methyl ester

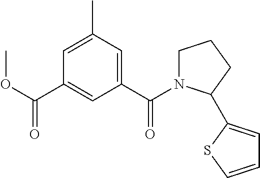

In analogy to the procedure described for the synthesis of N-Methyl-N-(4-methyl-thiazol-2-ylmethyl)-isophthalamic acid methyl ester the title compound was prepared from 5-Methyl-isophthalic acid monomethyl ester and 2-Thiophen-2-yl-pyrrolidine. MH⁺: 330.2.

### b) 3-Methyl-5-(2-thiophen-2-yl-pyrrolidine-1-carbonyl)-benzoic acid

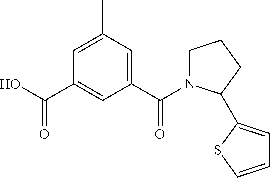

In analogy to the procedure described for the synthesis of N-Methyl-N-(4-methyl-thiazol-2-ylmethyl)-isophthalamic acid the title compound was prepared from 3-Methyl-5-(2-thiophen-2-yl-pyrrolidine-1-carbonyl)-benzoic acid methyl ester through cleavage of the ester. MH⁺: 316.1.

### c) N-{(1S,2R)-1-Benzyl-3-[(1-ethyl-1H-pyrazol-4-ylmethyl)-amino]-2-hydroxy-propyl}-3-methyl-5-(2-thiophen-2-yl-pyrrolidine-1-carbonyl)-benzamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 3-Methyl-5-(2-thiophen-2-yl-pyrrolidine-1-carbonyl)-benzoic acid. MH⁺: 586.6.

### Example 40

### N-{(1S,2R)-1-Benzyl-3-[(1-ethyl-1H-pyrazol-4-ylmethyl)-amino]-2-hydroxy-propyl}-5,N′-dimethyl-N′-(4-methyl-thiazol-2-ylmethyl)-isophthalamide

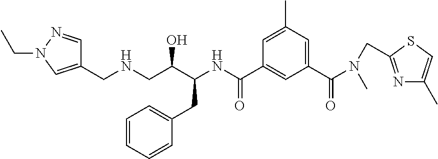

### a) 5,N-Dimethyl-N-(4-methyl-thiazol-2-ylmethyl)-isophthalamic acid methyl ester

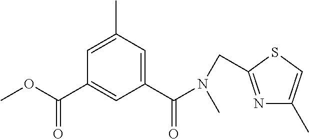

In analogy to the procedure described for the synthesis of N-Methyl-N-(4-methyl-thiazol-2-ylmethyl)-isophthalamic acid methyl ester the title compound was prepared from 5-Methyl-isophthalic acid monomethyl ester and Methyl-(4-methyl-thiazol-2-ylmethyl)-amine. MH⁺: 319.2.

### b) 5,N-Dimethyl-N-(4-methyl-thiazol-2-ylmethyl)-isophthalamic acid

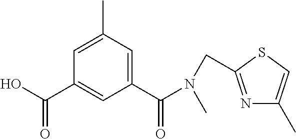

In analogy to the procedure described for the synthesis of N-Methyl-N-(4-methyl-thiazol-2-ylmethyl)-isophthalamic acid the title compound was prepared from 5,N-Dimethyl-N-(4-methyl-thiazol-2-ylmethyl)-isophthalamic acid methyl ester through cleavage of the ester. MH⁺: 305.1.

### c) N-{(1S,2R)-1-Benzyl-3-[(1-ethyl-1H-pyrazol-4-ylmethyl)-amino]-2-hydroxy-propyl}-5,N′-dimethyl-N′-(4-methyl-thiazol-2-ylmethyl)-isophthalamide

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-1-[(1-ethyl-1H-pyrazol-4-ylmethyl)-amino]-4-phenyl-butan-2-ol; hydrochloride and 5,N-Dimethyl-N-(4-methyl-thiazol-2-ylmethyl)-isophthalamic acid. MH⁺: 575.6.

### Example 41

### N-[(1S,2R)-1-Benzyl-2-hydroxy-3-(3-trifluoromethoxy-benzylamino)-propyl]-3-(methanesulfonyl-methyl-amino)-5-[(R)-2-(4-methyl-thiazol-2-yl)-pyrrolidine-1-carbonyl]-benzamide

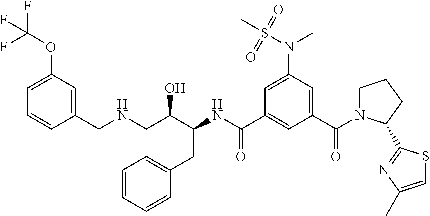

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(Methanesulfonyl-methyl-amino)-5-[(R)-2-(4-methyl-thiazol-2-yl)-pyrrolidine-1-carbonyl]-benzoic acid. MH⁺: 760.7.

### Example 42

### N-[(1S,2R)-1-Benzyl-2-hydroxy-3-(3-trifluoromethoxy-benzylamino)-propyl]-3-(methanesulfonyl-methyl-amino)-5-(2-phenyl-pyrrolidine-1-carbonyl)-benzamide

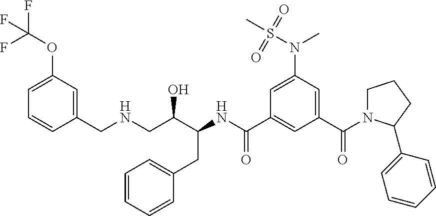

In analogy to the procedure described for the synthesis of N-[(1S,2R)-1-Benzyl-2-hydroxy-3-(3-trifluoromethyl-benzylamino)-propyl]-N′-methyl-N′-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(Methanesulfonyl-methyl-amino)-5-(2-phenyl-pyrrolidine-1-carbonyl)-benzoic acid. MH⁺: 739.7.

### Example 43

### N-[(1S,2R)-1-Benzyl-2-hydroxy-3-(3-trifluoromethoxy-benzylamino)-propyl]-3-methyl-5-(2-phenyl-pyrrolidine-1-carbonyl)-benzamide

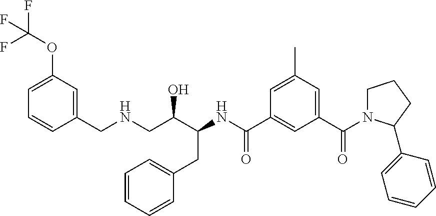

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-Methyl-5-(2-phenyl-pyrrolidine-1-carbonyl)-benzoic acid. MH⁺: 646.7.

### Example 44

### N-[(1S,2R)-1-Benzyl-2-hydroxy-3-(3-trifluoromethoxy-benzylamino)-propyl]-5,N′-dimethyl-N′-pyridin-2-ylmethyl-isophthalamide

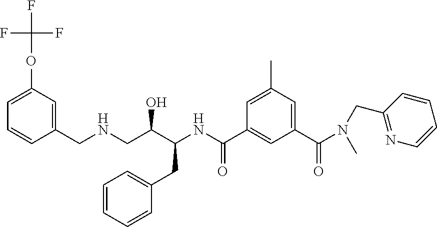

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 5,N-Dimethyl-N-pyridin-2-ylmethyl-isophthalamic acid. MH⁺: 621.6.

### Example 45

### N-[(1S,2R)-1-Benzyl-2-hydroxy-3-(3-trifluoromethoxy-benzylamino)-propyl]-3-(methanesulfonyl-methyl-amino)-5-(2-thiophen-2-yl-pyrrolidine-1-carbonyl)-benzamide

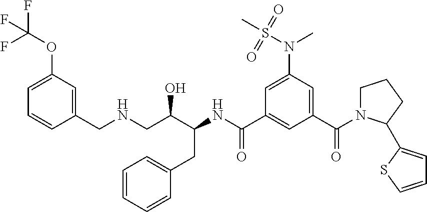

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-(Methanesulfonyl-methyl-amino)-5-(2-thiophen-2-yl-pyrrolidine-1-carbonyl)-benzoic acid. MH⁺: 745.7.

### Example 46

### N-[(1S,2R)-1-Benzyl-2-hydroxy-3-(3-trifluoromethoxy-benzylamino)-propyl]-3-methyl-5-(2-thiophen-2-yl-pyrrolidine-1-carbonyl)-benzamide

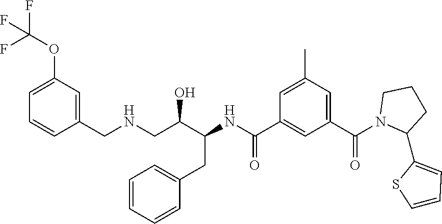

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 3-Methyl-5-(2-thiophen-2-yl-pyrrolidine-1-carbonyl)-benzoic acid. MH⁺: 652.6.

### Example 47

### N-[(1S,2R)-1-Benzyl-2-hydroxy-3-(3-trifluoromethoxy-benzylamino)-propyl]-5,N′-dimethyl-N′-(4-methyl-thiazol-2-ylmethyl)-isophthalamide

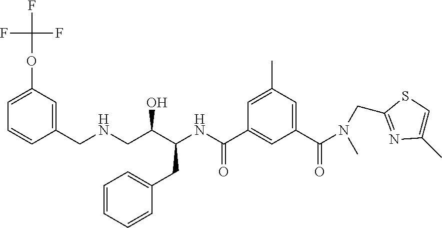

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethoxy-benzylamino)-butan-2-ol, hydrochloride and 5,N-Dimethyl-N-(4-methyl-thiazol-2-ylmethyl)-isophthalamic acid. MH⁺: 641.6.

### Example 48

### N-[(1S,2R)-1-Benzyl-2-hydroxy-3-(3-trifluoromethyl-benzylamino)-propyl]-3-(methanesulfonyl-methyl-amino)-5-[(R)-2-(4-methyl-thiazol-2-yl)-pyrrolidine-1-carbonyl]-benzamide

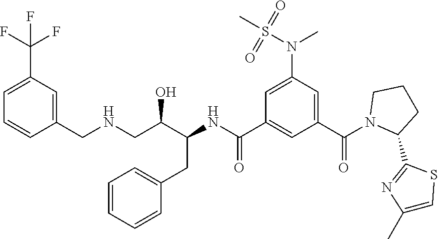

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-(Methanesulfonyl-methyl-amino)-5-[(R)-2-(4-methyl-thiazol-2-yl)-pyrrolidine-1-carbonyl]-benzoic acid. MH⁺: 744.7.

### Example 49

### N-[(1S,2R)-1-Benzyl-2-hydroxy-3-(3-trifluoromethyl-benzylamino)-propyl]-3-methyl-5-[(R)-2-(4-methyl-thiazol-2-yl)-pyrrolidine-1-carbonyl]-benzamide

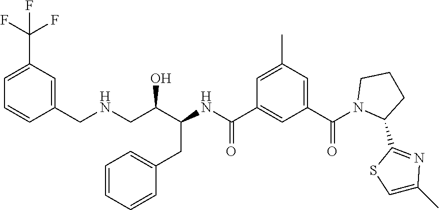

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-Methyl-5-[(R)-2-(4-methyl-thiazol-2-yl)-pyrrolidine-1-carbonyl]-benzoic acid. MH⁺: 651.6.

### Example 51

### N-[(1S,2R)-1-Benzyl-2-hydroxy-3-(3-trifluoromethyl-benzylamino)-propyl]-3-methyl-5-(2-phenyl-pyrrolidine-1-carbonyl)-benzamide

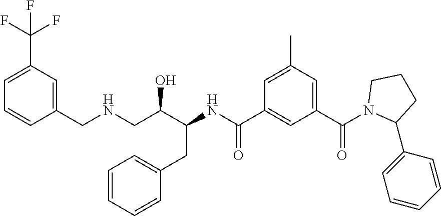

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-Methyl-5-(2-phenyl-pyrrolidine-1-carbonyl)-benzoic acid. MH⁺: 630.6.

### Example 52

### N-[(1S,2R)-1-Benzyl-2-hydroxy-3-(3-trifluoromethyl-benzylamino)-propyl]-5,N′-dimethyl-N′-pyridin-2-ylmethyl-isophthalamide

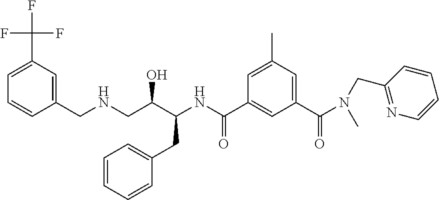

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 5,N-Dimethyl-N-pyridin-2-ylmethyl-isophthalamic acid. MH⁺: 605.6.

### Example 53

### N-[(1S,2R)-1-Benzyl-2-hydroxy-3-(3-trifluoromethyl-benzylamino)-propyl]-3-(methanesulfonyl-methyl-amino)-5-(2-thiophen-2-yl-pyrrolidine-1-carbonyl)-benzamide

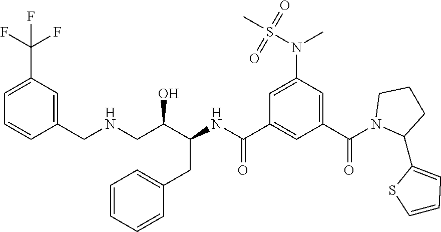

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-(Methanesulfonyl-methyl-amino)-5-(2-thiophen-2-yl-pyrrolidine-1-carbonyl)-benzoic acid. MH⁺: 729.7.

### Example 54

### N-[(1S,2R)-1-Benzyl-2-hydroxy-3-(3-trifluoromethyl-benzylamino)-propyl]-3-methyl-5-(2-thiophen-2-yl-pyrrolidine-1-carbonyl)-benzamide

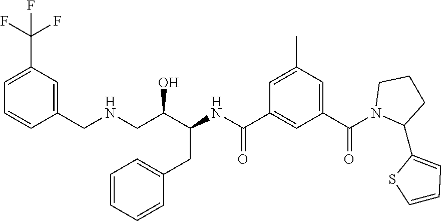

In analogy to the procedure described for the synthesis of N-[(1S,2R)-1-Benzyl-2-hydroxy-3-(3-trifluoromethyl-benzylamino)-propyl]-N-methyl-N-(4-methyl-thiazol-2-ylmethyl)-isophthalamide the title compound was prepared from (2R,3S)-3-Amino-4-phenyl-1-(3-trifluoromethyl-benzylamino)-butan-2-ol, hydrochloride and 3-Methyl-5-(2-thiophen-2-yl-pyrrolidine-1-carbonyl)-benzoic acid. MH⁺: 636.6.

### Example 55

### N-[(1S,2R)-1-Benzyl-2-hydroxy-3-(3-trifluoromethyl-benzylamino)-propyl]-3-(2-p-tolyl-pyrrolidine-1-carbonyl)-benzamide

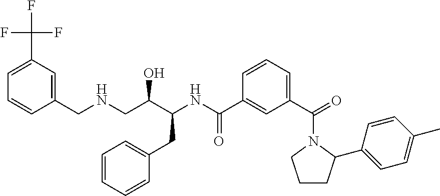

### a) N-[(1S,2R)-1-Benzyl-2-hydroxy-3-(3-trifluoromethyl-benzylamino)-propyl]-iso phthalamic acid methyl ester

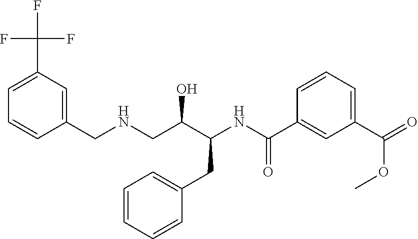

A mixture of isophthalic acid monomethyl ester (396 mg, 2.2 mmol) and TBTU (861 mg, 2.6 mmol) in DMF (5 mL) was stirred at 22° C. for 30 min. (2R,3S)-3-Amino-4-phenyl-1-(3-trifluoromethyl-benzylamino)-butan-2-ol, dihydrochloride (823 mg, 2.00 mmol) and DIPEA (775 mg, 6 mmol) were then added to the activated esters. The reaction mixture was stirred at 22° C. for 5 hr and then poured into 1 M NaHCO₃ and extracted with AcOEt (2×15 mL). The organic layers were combined and concentrated in vacuo and used in the consecutive step without further purification. MH⁺: 501.4.

### b) N-[(1S,2R)-1-Benzyl-2-hydroxy-3-(3-trifluoromethyl-benzylamino)-propyl]-isophthalamic acid

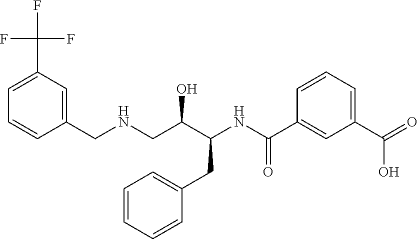

N-[(1S,2R)-1-Benzyl-2-hydroxy-3-(3-trifluoromethyl-benzylamino)-propyl]-isophthalamic acid methyl ester was treated with LiOH.H₂O (336 mg, 80 mmol) at 22° C. for 16 hr in a mixture of THF (8 mL)/water (2 mL) and then concentrated in vacuo. The crude materials were purified by preparative HPLC on reversed phase eluting with a gradient formed from acetonitrile, water and formic acid. The product containing fractions were evaporated to yield the title compound. MH⁺: 487.1.

### c) N-[(1S,2R)-1-Benzyl-2-hydroxy-3-(3-trifluoromethyl-benzylamino)-propyl]-3-(2-p-tolyl-pyrrolidine-1-carbonyl)-benzamide

A mixture of N-[(1S,2R)-1-Benzyl-2-hydroxy-3-(3-trifluoromethyl-benzylamino)-propyl]-isophthalamic acid (48.6 mg, 0.1 mmol) and TBTU (43 mg, 0.13 mmol) in DMF (1.5 mL) was stirred at 22° C. for 30 min. 2-p-tolylpyrrolidine (19.3 mg, 0.12 mmol) and DIPEA (25.8 mg, 0.2 mmol) was then added to the activated ester. The reaction mixture was stirred at 22° C. for 16 hr. The crude reaction mixtures were concentrated in vacuo and purified by preparative HPLC on reversed phase eluting with a gradient formed from acetonitrile, water and formic acid. The product containing fractions were evaporated to yield 16 mg (26%) of the title compound. MH⁺: 630.5.

### Example 56

### N-[(1S,2R)-1-Benzyl-2-hydroxy-3-(3-trifluoromethyl-benzylamino)-propyl]-3-[2-(4-methoxy-phenyl)-pyrrolidine-1-carbonyl]-benzamide

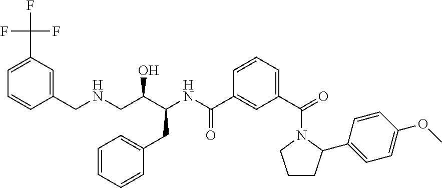

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(4-Methoxy-phenyl)-pyrrolidine. MH⁺: 646.5.

### Example 57

### N-[(1S,2R)-1-Benzyl-2-hydroxy-3-(3-trifluoromethyl-benzylamino)-propyl]-3-[2-(3-chloro-phenyl)-pyrrolidine-1-carbonyl]-benzamide

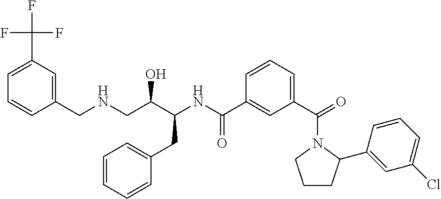

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(3-Chloro-phenyl)-pyrrolidine. MH⁺: 650.3.

### Example 58

### N-[(1S,2R)-1-Benzyl-2-hydroxy-3-(3-trifluoromethyl-benzylamino)-propyl]-3-(2-m-tolyl-pyrrolidine-1-carbonyl)-benzamide

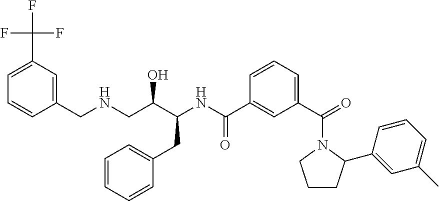

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-m-Tolyl-pyrrolidine. MH⁺: 630.5.

### Example 59

### N-[(1S,2R)-1-Benzyl-2-hydroxy-3-(3-trifluoromethyl-benzylamino)-propyl]-3-[2-(3-chloro-4-fluoro-phenyl)-pyrrolidine-1-carbonyl]-benzamide

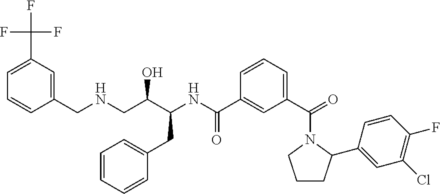

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(3-Chloro-4-fluoro-phenyl)-pyrrolidine. MH⁺: 668.1.

### Example 60

### N-[(1S,2R)-1-Benzyl-2-hydroxy-3-(3-trifluoromethyl-benzylamino)-propyl]-3-[2-(4-chloro-3-methyl-phenyl)-pyrrolidine-1-carbonyl]-benzamide

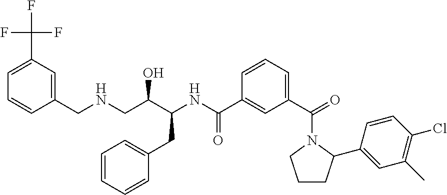

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(4-Chloro-3-methyl-phenyl)-pyrrolidine. MH⁺: 664.5.

### Example 61

### N-[(1S,2R)-1-Benzyl-2-hydroxy-3-(3-trifluoromethyl-benzylamino)-propyl]-3-[2-(2,4-dimethyl-phenyl)-pyrrolidine-1-carbonyl]-benzamide

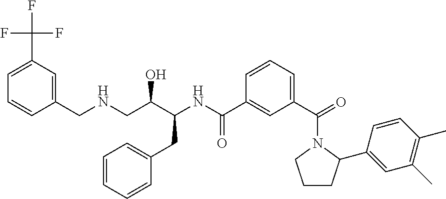

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(2,4-Dimethyl-phenyl)-pyrrolidine. MH⁺: 644.5.

### Example 62

### N-[(1S,2R)-1-Benzyl-2-hydroxy-3-(3-trifluoromethyl-benzylamino)-propyl]-3-[2-(4-ethyl-phenyl)-pyrrolidine-1-carbonyl]-benzamide

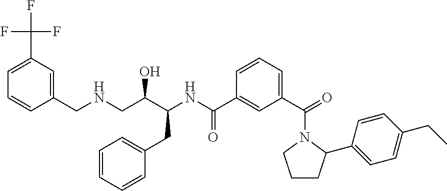

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(4-Ethyl-phenyl)-pyrrolidine. MH⁺: 644.6.

### Example 63

### N-[(1S,2R)-1-Benzyl-2-hydroxy-3-(3-trifluoromethyl-benzylamino)-propyl]-3-[2-(4-fluoro-phenyl)-3-methyl-pyrrolidine-1-carbonyl]-benzamide

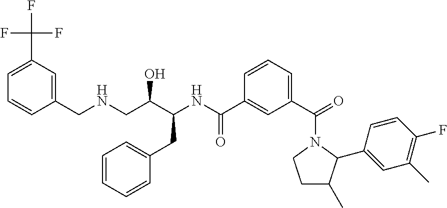

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(4-Fluoro-phenyl)-3-methyl-pyrrolidine. MH⁺: 648.5.

### Example 64

### 3-[4-Acetyl-2-(4-fluoro-phenyl)-pyrrolidine-1-carbonyl]-N-[(1S,2R)-1-benzyl-2-hydroxy-3-(3-trifluoromethyl-benzylamino)-propyl]-benzamide

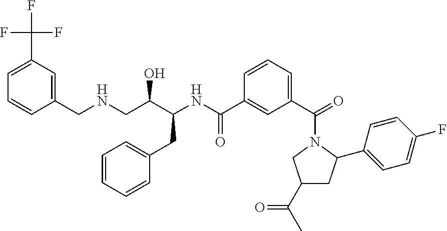

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 1-[5-(4-Fluoro-phenyl)-pyrrolidin-3-yl]-ethanone. MH⁺: 676.5.

### Example 65

### N-[(1S,2R)-1-Benzyl-2-hydroxy-3-(3-trifluoromethyl-benzylamino)-propyl]-3-((S)-2-benzyl-pyrrolidine-1-carbonyl)-benzamide

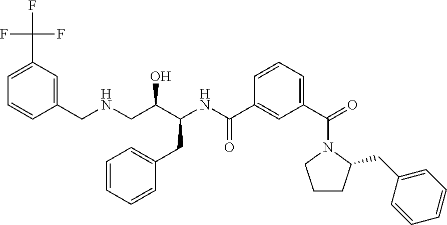

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and (S)-2-Benzyl-pyrrolidine. MH⁺: 630.5.

### Example 66

### N-[(1S,2R)-1-Benzyl-2-hydroxy-3-(3-trifluoromethyl-benzylamino)-propyl]-3-((R)-2-phenyl-pyrrolidine-1-carbonyl)-benzamide

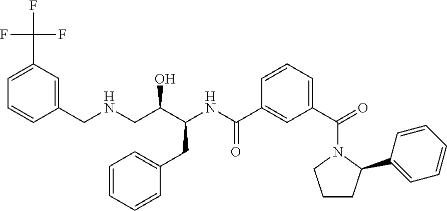

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and (R)-2-Phenyl-pyrrolidine. MH⁺: 616.5.

### Example 67

### N-[(1S,2R)-1-Benzyl-2-hydroxy-3-(3-trifluoromethyl-benzylamino)-propyl]-3-[(S)-2-(4-fluoro-phenyl)-pyrrolidine-1-carbonyl]-benzamide

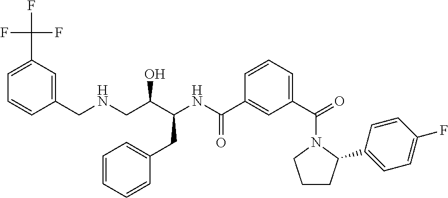

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and (S)-2-(4-Fluoro-phenyl)-pyrrolidine. MH⁺: 634.5.

### Example 68

### N-[(1S,2R)-1-Benzyl-2-hydroxy-3-(3-trifluoromethyl-benzylamino)-propyl]-3-[2-(3-fluoro-phenyl)-pyrrolidine-1-carbonyl]-benzamide

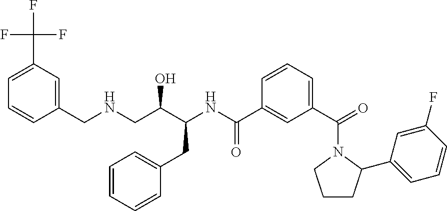

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-(3-Fluoro-phenyl)-pyrrolidine. MH⁺: 634.6.

### Example 69

### N-[(1S,2R)-1-Benzyl-2-hydroxy-3-(3-trifluoromethyl-benzylamino)-propyl]-3-(2-cyclohexyl-pyrrolidine-1-carbonyl)-benzamide

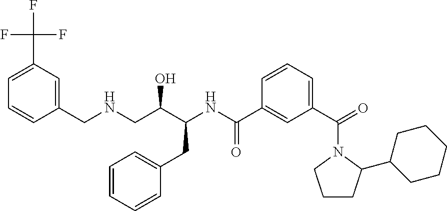

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-Cyclohexyl-pyrrolidine. MH⁺: 622.7.

### Example 70

### N-[(1S,2R)-1-Benzyl-2-hydroxy-3-(3-trifluoromethyl-benzylamino)-propyl]-3-(2-isobutyl-pyrrolidine-1-carbonyl)-benzamide

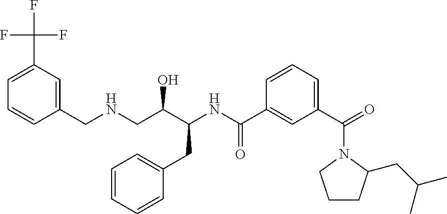

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-Isobutyl-pyrrolidine. MH⁺: 596.6.

### Example 71

### 3-(2-Benzoyl-pyrrolidine-1-carbonyl)-N-[(1S,2R)-1-benzyl-2-hydroxy-3-(3-trifluoro methyl-benzylamino)-propyl]-benzamide

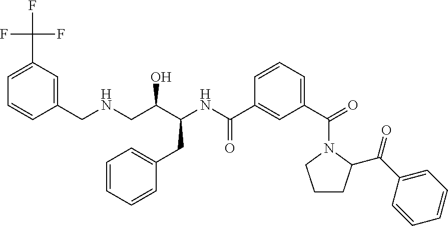

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and Phenyl-pyrrolidin-2-yl-methanone. MH⁺: 644.6.

### Example 72

### N-[(1S,2R)-1-Benzyl-2-hydroxy-3-(3-trifluoromethyl-benzylamino)-propyl]-3-(2-cyclopentyl-pyrrolidine-1-carbonyl)-benzamide

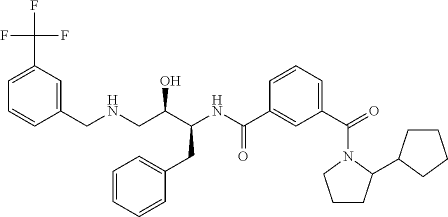

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-Cyclopentyl-pyrrolidine. MH⁺: 608.6.

### Example 73

### N-[(1S,2R)-1-Benzyl-2-hydroxy-3-(3-trifluoromethyl-benzylamino)-propyl]-3-(2-tert-butyl-pyrrolidine-1-carbonyl)-benzamide

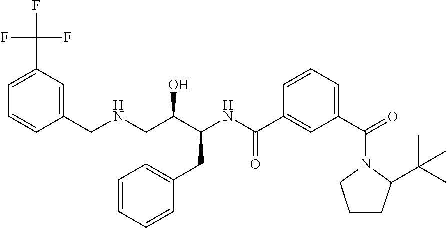

In analogy to the procedure described for the synthesis of N-[(1S,2R)-1-Benzyl-2-hydroxy-3-(3-trifluoromethyl-benzylamino)-propyl]-3-(2-p-tolyl-pyrrolidine-1-carbonyl)-benzamide the title compound was prepared from N-[(1S,2R)-1-Benzyl-2-hydroxy-3-(3-trifluoromethyl-benzylamino)-propyl]-isophthalamic acid and 2-tert-Butyl-pyrrolidine. MH⁺: 596.6.

### Example 74

### N-((1S,2R)-1-Benzyl-3-{[5-(1-fluoro-1-methyl-ethyl)-pyridin-3-ylmethyl]-amino}-2-hydroxy-propyl)-5-(methanesulfonyl-methyl-amino)-N′-methyl-N′-(4-methyl-thiazol-2-ylmethyl)-isophthalamide

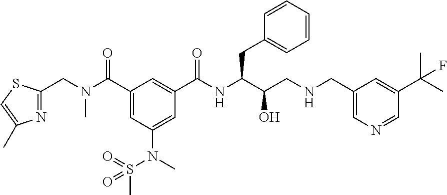

The title compound was prepared in analogy to the procedure described in WO 2009/015 369. MH⁺: 711.7.

### Example 75

### Assay for RACE Inhibition by Measuring Cellular TMEM27 Cleavage

This assay uses the principle of inhibition of human TMEM27 cleavage by endogenous cellular BACE2 in the Ins1e rat cell line and shedding from the cell surface into the culture medium, followed by detection in an ELISA assay. Inhibition of BACE2 prevents the cleavage and shedding in a dose-dependent manner.

The stable cell line “INS-TMEM27” represents an INS1e-derived cell line with inducible expression (using the TetOn system) of full-length hTMEM27 in a doxycycline-dependent manner. The cells were cultured throughout the experiment in RPMI1640+Glutamax (Invitrogen) Penicillin/Streptomycin, 10% Fetal bovine serum, 100 mM pyruvate, 5 mM beta-mercatptoethanol, 100 micrograms/ml G418 and 100 microgram/ml hygromycin and were grown in adherent culture at 37° C. in a standard CO₂ cell culture incubator.

INS-TMEM27 cells were seeded in 96-well plates. After 2 days in culture, BACE2 inhibitor was added in a range of concentrations as required by the assay and after a further two hours, doxycycline was added to a final concentration of 500 ng/ml. The cells were incubated for a further 46 hours and the supernatant harvested for detection of shed TMEM27.

An ELISA assay (using a pair of mouse anti-human-TMEM27 antibodies, raised against the extracellular domain of TMEM27) was used for detection of TMEM27 in the culture medium. An IC₅₀ for BACE2 inhibition was calculated using the ELISA readout for each inhibitor concentration with standard curve-fitting software.

The inhibitory activity, given as an IC₅₀ value, of the compounds of Examples 1 to 32 and 34 to 74 were as follows.

| Ex. | BACE2 IC₅₀ [μM] |
|---|---|
| 1 | 0.205 |
| 2 | 0.258 |
| 3 | 0.175 |
| 4 | 0.471 |
| 5 | 0.140 |
| 6 | 0.520 |
| 7 | 0.478 |
| 8 | 0.822 |
| 9 | 0.263 |
| 10 | 0.014 |
| 11 | 0.009 |
| 12 | 0.075 |
| 13 | 0.068 |
| 14 | 0.063 |
| 15 | 0.332 |
| 16 | 0.055 |
| 17 | 0.213 |
| 18 | 0.047 |
| 19 | 0.008 |
| 20 | 0.143 |
| 21 | 0.037 |
| 22 | 0.0312 |
| 23 | 0.0577 |
| 24 | 0.068 |
| 25 | 0.070 |
| 26 | 0.284 |
| 27 | 0.314 |
| 28 | 0.511 |
| 29 | 0.112 |
| 30 | 0.016 |
| 31 | 0.239 |
| 32 | 0.138 |
| 34 | 0.145 |
| 35 | 0.0002 |
| 36 | 0.001 |
| 37 | 0.001 |
| 38 | 0.0003 |
| 39 | 0.001 |
| 40 | 0.104 |
| 41 | 0.0001 |
| 42 | 0.062 |
| 43 | 0.003 |
| 44 | 0.049 |
| 45 | 0.011 |
| 46 | 0.001 |
| 47 | 0.117 |
| 48 | 0.081 |
| 49 | 0.119 |
| 50 | 0.003 |
| 51 | 0.0004 |
| 52 | 0.095 |
| 53 | 0.029 |
| 54 | 0.0019 |
| 55 | 0.0420 |
| 56 | 0.1150 |
| 57 | 0.619 |
| 58 | 0.089 |
| 59 | 0.033 |
| 60 | 0.267 |
| 61 | 0.2220 |
| 62 | 0.764 |
| 63 | 0.001 |
| 64 | 0.026 |
| 65 | 0.051 |
| 66 | 0.314 |
| 67 | 0.004 |
| 68 | 0.002 |
| 69 | 0.007 |
| 70 | 0.147 |
| 71 | 0.484 |
| 72 | 0.066 |
| 73 | 0.056 |
| 74 | 0.255 |

1. A method for the treatment of diabetes comprising the step of administering to a patient in need thereof a compound of formula I, 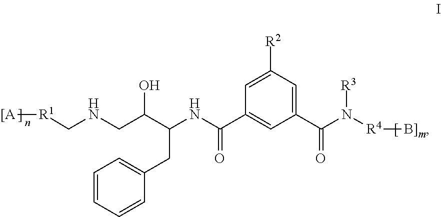
wherein
R¹ is selected from the group consisting of
i) aryl, and
ii) heteroaryl,
R² is selected from the group consisting of
i) H,
ii) C₁₋₆-alkyl, and
iii) —N(R⁵,R⁶);
R³ is selected from the group consisting of
i) H, and
ii) C₁₋₆-alkyl,
R⁴ is selected from the group consisting of
i) —CH₂-aryl, and
ii) —CH₂-heteroaryl;
or R³ and R⁴ together with the nitrogen to which they are attached form a five membered heterocyclyl, optionally substituted by Z, or 3-aza-bicyclo[3.2.1]octane-3-yl, optionally substituted by C₁₋₆-alkyl;
R⁵ is selected from the group consisting of
i) H, and
ii) C₁₋₆-alkyl;
R⁶ is —SO₂—C₁₋₆-alkyl;
n is 0, 1 or 2;
m is 0, 1 or 2;
A is independently selected from the group consisting of
i) acetamidyl,
ii) acetyl,
iii) amido,
iv) amino,
v) C₁₋₆-alkoxy,
vi) C₁₋₆-alkyl,
vii) carboxy,
viii) cyano,
ix) halogen,
x) halogen-C₁₋₆-alkoxy,
xi) halogen-C₁₋₆-alkyl,
xii) hydroxy,
xiii) —N(C₁₋₆-alkyl, C₁₋₆-alkyl),
xiv) —N(H, C₁₋₆-alkyl), and
xv) —SO₂—C₁₋₆-alkyl
B is C₁₋₆-alkyl; and
Z is independently selected from the group consisting of
i) aryl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
ii) heteroaryl, optionally substituted by 1 or 2 C₁₋₆-alkyl,
iii) cycloalkyl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
iv) acetyl,
v) benzoyl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro,
vi) benzyl, optionally substituted by 1 or 2 substituents individually selected from the group consisting of acetamidyl, acetyl, amido, amino, carboxy, cyano, halogen, halogen-C₁₋₆-alkoxy, halogen-C₁₋₆-alkyl, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy, C₁₋₆-alkyl and nitro, and
vii) C₁₋₆-alkyl, optionally substituted by 1 or 2 substituents individually selected from the group consisting of amino, cyano, halogen, hydroxy, (C₁₋₆-alkyl,H)N—, (C₁₋₆-alkyl, C₁₋₆-alkyl)N—, C₁₋₆-alkyl-S(O)₂—, C₁₋₆-alkoxy and nitro,
or a pharmaceutically acceptable salt thereof.

2. A method according to claim 1, wherein
R¹ is selected from the group consisting of
i) phenyl,
ii) pyrazolyl, and
iii) pyridinyl;
R² is selected from the group consisting of
i) H,
ii) C₁₋₆-alkyl, and
iii) —N(R⁵,R⁶);
R³ is selected from the group consisting of
i) H, and
ii) C₁₋₆-alkyl,
R⁴ is selected from the group consisting of
i) —CH₂-pyridinyl, and
ii) —CH₂-thiazolyl
or R³ and R⁴ together with the nitrogen to which they are attached form pyrrolidinyl, optionally substituted by Z, or 3-aza-bicyclo[3.2.1]octane-3-yl, optionally substituted by C₁₋₆-alkyl;
R⁵ is selected from the group consisting of
i) H, and
ii) C₁₋₆-alkyl;
R⁶ is —SO₂—C₁₋₆-alkyl;
n is 0 or 1;
m is 0, 1 or 2;
A is independently selected from the group consisting of
i) C₁₋₆-alkyl,
ii) halogen-C₁₋₆-alkyl,
iii) C₁₋₆-alkoxy, and
iv) halogen-C₁₋₆-alkoxy;
B is C₁₋₆-alkyl; and
Z is independently selected from the group consisting of
i) pyridinyl, optionally substituted by C₁₋₆-alkyl,
ii) thiazolyl, optionally substituted by C₁₋₆-alkyl,
iii) pyrazolyl, optionally substituted by C₁₋₆-alkyl,
iv) isoxazolyl, optionally substituted by C₁₋₆-alkyl,
v) [1,2,4]oxadiazol, optionally substituted by C₁₋₆-alkyl,
vi) thiophenyl, optionally substituted by C₁₋₆-alkyl,
vii) cyclopentyl, optionally substituted by C₁₋₆-alkyl
viii) cyclohexyl, optionally substituted by C₁₋₆-alkyl,
ix) benzoyl, optionally substituted by C₁₋₆-alkyl
x) benzyl, optionally substituted by 1 or 2 substituents independently selected from the group consisting of C₁₋₆-alkyl, halo and C₁₋₆-alkoxy
xi) phenyl, optionally substituted by 1 or 2 substituents independently selected from the group consisting of C₁₋₆-alkyl, halo and C₁₋₆-alkoxy; and
xii) C₁₋₆-alkyl;
or a pharmaceutically acceptable salt thereof.

3. A method according to claim 1, wherein R¹ is phenyl.

4. A method according to claim 1, wherein R² is H or N(methyl,methanesulfonyl).

5. A method according to claim 1, wherein R³ is methyl and R⁴ is —CH₂-thiazolyl, optionally substituted by methyl.

6. A method according to claim 1, wherein R³ and R⁴ together with the nitrogen to which they are attached form pyrrolidinyl, optionally substituted by methyl-thiazolyl, phenyl, thiophenyl, fluoro-phenyl, methyl cyclohexyl or cyclopentyl.

7. A compound of formula I, which is a compound of formula Ia 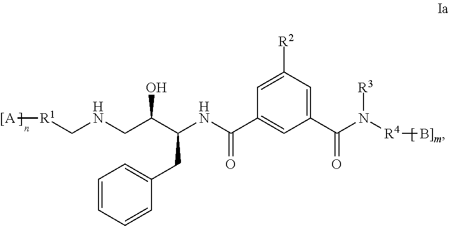
selected from the group consisting of
N-[(1S,2R)-1-Benzyl-2-hydroxy-3-(3-trifluoromethoxy-benzylamino)-propyl]-3-(2-pyridin-4-yl-pyrrolidine-1-carbonyl)-benzamide,
N-[(1S,2R)-1-Benzyl-2-hydroxy-3-(3-trifluoromethyl-benzylamino)-propyl]-3-(2-pyridin-4-yl-pyrrolidine-1-carbonyl)-benzamide,
N-[(1S,2R)-1-Benzyl-2-hydroxy-3-(3-trifluoromethoxy-benzylamino)-propyl]-N-pyridin-2-ylmethyl-isophthalamide,
N-[(1S,2R)-1-Benzyl-2-hydroxy-3-(3-trifluoromethyl-benzylamino)-propyl]-N-pyridin-2-ylmethyl-isophthalamide,
N-[(1S,2R)-1-Benzyl-2-hydroxy-3-(3-trifluoromethoxy-benzylamino)-propyl]-3-((1R,5S)-1,8,8-trimethyl-3-aza-bicyclo[3.2.1]octane-3-carbonyl)-benzamide,
N-[(1S,2R)-1-Benzyl-2-hydroxy-3-(3-trifluoromethoxy-benzylamino)-propyl]-3-(3-pyridin-2-yl-pyrrolidine-1-carbonyl)-benzamide,
N-[(1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide,
N-[(1S,2R)-1-Benzyl-2-hydroxy-3-(3-trifluoromethyl-benzylamino)-propyl]-3-(2-phenyl-pyrrolidine-1-carbonyl)-benzamide,
N-[(1S,2R)-1-Benzyl-2-hydroxy-3-(3-trifluoromethyl-benzylamino)-propyl]-3-(2-pyridin-3-yl-pyrrolidine-1-carbonyl)-benzamide, and
N-[(1S,2R)-1-Benzyl-2-hydroxy-3-(3-trifluoromethyl-benzylamino)-propyl]-3-[2-(1-methyl-1H-pyrazol-3-yl)-pyrrolidine-1-carbonyl]-benzamide,
or a pharmaceutically acceptable salt thereof.

8. A compound according to claim 7, wherein the compound is selected from the group consisting of
N-[(1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide,
N-[(1S,2R)-1-Benzyl-2-hydroxy-3-(3-trifluoromethyl-benzylamino)-propyl]-3-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-methyl-5-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-methyl-5-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-3-(methanesulfonyl-methyl-amino)-5-(2-phenyl-pyrrolidine-1-carbonyl)-benzamide,
N-[(1S,2R)-1-Benzyl-2-hydroxy-3-(3-trifluoromethoxy-benzylamino)-propyl]-3-methyl-5-(2-phenyl-pyrrolidine-1-carbonyl)-benzamide,
N-[(1S,2R)-1-Benzyl-2-hydroxy-3-(3-trifluoromethoxy-benzylamino)-propyl]-3-(methanesulfonyl-methyl-amino)-5-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-3-methyl-5-(2-thiophen-2-yl-pyrrolidine-1-carbonyl)-benzamide,
N-[(1S,2R)-1-Benzyl-2-hydroxy-3-(3-trifluoromethyl-benzylamino)-propyl]-3-(methanesulfonyl-methyl-amino)-5-(2-phenyl-pyrrolidine-1-carbonyl)-benzamide,
N-[(1S,2R)-1-Benzyl-2-hydroxy-3-(3-trifluoromethyl-benzylamino)-propyl]-3-methyl-5-(2-thiophen-2-yl-pyrrolidine-1-carbonyl)-benzamide,
N-[(1S,2R)-1-Benzyl-2-hydroxy-3-(3-trifluoromethyl-benzylamino)-propyl]-3-[2-(4-fluoro-phenyl)-3-methyl-pyrrolidine-1-carbonyl]-benzamide,
N-[(1S,2R)-1-Benzyl-2-hydroxy-3-(3-trifluoromethyl-benzylamino)-propyl]-3-(2-cyclohexyl-pyrrolidine-1-carbonyl)-benzamide, and
N-[(1S,2R)-1-Benzyl-2-hydroxy-3-(3-trifluoromethyl-benzylamino)-propyl]-3-(2-cyclopentyl-pyrrolidine-1-carbonyl)-benzamide,
or a pharmaceutically acceptable salt thereof.

9. A compound according to claim 8, wherein the compound is selected from the group consisting of
N-[(1S,2R)-1-Benzyl-2-hydroxy-3-(3-trifluoromethoxy-benzylamino)-propyl]-3-methyl-5-[(R)-2-(4-methyl-thiazol-2-yl)-pyrrolidine-1-carbonyl]-benzamide,
N-[(1S,2R)-1-Benzyl-2-hydroxy-3-(3-trifluoromethyl-benzylamino)-propyl]-3-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-3-(methanesulfonyl-methyl-amino)-5-(2-phenyl-pyrrolidine-1-carbonyl)-benzamide,
N-[(1S,2R)-1-Benzyl-2-hydroxy-3-(3-trifluoromethoxy-benzylamino)-propyl]-3-methyl-5-(2-phenyl-pyrrolidine-1-carbonyl)-benzamide,
N-[(1S,2R)-1-Benzyl-2-hydroxy-3-(3-trifluoromethoxy-benzylamino)-propyl]-3-(methanesulfonyl-methyl-amino)-5-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-3-methyl-5-(2-thiophen-2-yl-pyrrolidine-1-carbonyl)-benzamide, and
N-[(1S,2R)-1-Benzyl-2-hydroxy-3-(3-trifluoromethyl-benzylamino)-propyl]-3-(2-cyclopentyl-pyrrolidine-1-carbonyl)-benzamide,
or a pharmaceutically acceptable salt thereof.

10. A compound according to claim 7,
wherein the compound is selected from the group consisting of
N-[(1S,2R)-1-Benzyl-2-hydroxy-3-(3-trifluoromethyl-benzylamino)-propyl]-3-[2-(2-methyl-2H-pyrazol-3-yl)-pyrrolidine-1-carbonyl]-benzamide,
N-[(1S,2R)-1-Benzyl-2-hydroxy-3-(3-trifluoromethyl-benzylamino)-propyl]-3-[2-(3-methyl-isoxazol-5-yl)-pyrrolidine-1-carbonyl]-benzamide,
N-[(1S,2R)-1-Benzyl-2-hydroxy-3-(3-trifluoromethyl-benzylamino)-propyl]-3-[2-(3-methyl-[1,2,4]oxadiazol-5-yl)-pyrrolidine-1-carbonyl]-benzamide,
N-[(1S,2R)-1-Benzyl-2-hydroxy-3-(3-trifluoromethyl-benzylamino)-propyl]-N′-methyl-N′-pyridin-2-ylmethyl-isophthalamide,
N-[(1S,2R)-1-Benzyl-2-hydroxy-3-(3-trifluoromethyl-benzylamino)-propyl]-3-(2-thiophen-2-yl-pyrrolidine-1-carbonyl)-benzamide,
N-[(1S,2R)-1-Benzyl-2-hydroxy-3-(3-methoxy-benzylamino)-propyl]-3-[(R)-2-(4-methyl-thiazol-2-yl)-pyrrolidine-1-carbonyl]-benzamide,
N-[(1S,2R)-1-Benzyl-2-hydroxy-3-(3-trifluoromethoxy-benzylamino)-propyl]-3-[(R)-2-(4-methyl-thiazol-2-yl)-pyrrolidine-1-carbonyl]-benzamide,
N-[(1S,2R)-1-Benzyl-2-hydroxy-3-(3-methoxy-benzylamino)-propyl]-3-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(2-phenyl-pyrrolidine-1-carbonyl)-benzamide, and
N-[(1S,2R)-1-Benzyl-2-hydroxy-3-(3-trifluoromethoxy-benzylamino)-propyl]-3-(2-phenyl-pyrrolidine-1-carbonyl)-benzamide,
or a pharmaceutically acceptable salt thereof.

11. A compound according to claim 7,
wherein the compound is selected from the group consisting of
N-[(1S,2R)-1-Benzyl-2-hydroxy-3-(3-trifluoromethoxy-benzylamino)-propyl]-3-(2-pyridin-3-yl-pyrrolidine-1-carbonyl)-benzamide,
N-[(1S,2R)-1-Benzyl-2-hydroxy-3-(3-trifluoromethoxy-benzylamino)-propyl]-3-[2-(3-methyl-isoxazol-5-yl)-pyrrolidine-1-carbonyl]-benzamide,
N-[(1S,2R)-1-Benzyl-2-hydroxy-3-(3-methoxy-benzylamino)-propyl]-3-[2-(5-methyl-thiophen-2-yl)-pyrrolidine-1-carbonyl]-benzamide,
N-[(1S,2R)-1-Benzyl-2-hydroxy-3-(3-trifluoromethoxy-benzylamino)-propyl]-N-methyl-N-pyridin-2-ylmethyl-isophthalamide,
N-[(1S,2R)-1-Benzyl-2-hydroxy-3-(3-methoxy-benzylamino)-propyl]-3-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-3-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-[(R)-2-(4-methyl-thiazol-2-yl)-pyrrolidine-1-carbonyl]-benzamide,
N-{(1S,2R)-1-Benzyl-3-[(1-ethyl-1H-pyrazol-4-ylmethyl)-amino]-2-hydroxy-propyl}-3-methyl-5-[(R)-2-(4-methyl-thiazol-2-yl)-pyrrolidine-1-carbonyl]-benzamide, and
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-phenyl-pyrrolidine-1-carbonyl)-benzamide,
or a pharmaceutically acceptable salt thereof.

12. A compound according to claim 7,
wherein the compound is selected from the group consisting of
N-{(1S,2R)-1-Benzyl-3-[(1-ethyl-1H-pyrazol-4-ylmethyl)-amino]-2-hydroxy-propyl}-3-methyl-5-(2-phenyl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-5,N′-dimethyl-N′-pyridin-2-ylmethyl-isophthalamide,
N-{(1S,2R)-1-Benzyl-3-[(1-ethyl-1H-pyrazol-4-ylmethyl)-amino]-2-hydroxy-propyl}-3-(methanesulfonyl-methyl-amino)-5-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-3-methyl-5-(2-thiophen-2-yl-pyrrolidine-1-carbonyl)-benzamide,
N-{(1S,2R)-1-Benzyl-3-[(1-ethyl-1H-pyrazol-4-ylmethyl)-amino]-2-hydroxy-propyl}-5,N′-dimethyl-N′-(4-methyl-thiazol-2-ylmethyl)-isophthalamide,
N-[(1S,2R)-1-Benzyl-2-hydroxy-3-(3-trifluoromethoxy-benzylamino)-propyl]-3-(methanesulfonyl-methyl-amino)-5-[(R)-2-(4-methyl-thiazol-2-yl)-pyrrolidine-1-carbonyl]-benzamide,
N-[(1S,2R)-1-Benzyl-2-hydroxy-3-(3-trifluoromethoxy-benzylamino)-propyl]-3-(methanesulfonyl-methyl-amino)-5-(2-phenyl-pyrrolidine-1-carbonyl)-benzamide,
N-[(1S,2R)-1-Benzyl-2-hydroxy-3-(3-trifluoromethoxy-benzylamino)-propyl]-3-methyl-5-(2-phenyl-pyrrolidine-1-carbonyl)-benzamide,
N-[(1S,2R)-1-Benzyl-2-hydroxy-3-(3-trifluoromethoxy-benzylamino)-propyl]-5,N′-dimethyl-N′-pyridin-2-ylmethyl-isophthalamide, and
N-[(1S,2R)-1-Benzyl-2-hydroxy-3-(3-trifluoromethoxy-benzylamino)-propyl]-3-(methanesulfonyl-methyl-amino)-5-(2-thiophen-2-yl-pyrrolidine-1-carbonyl)-benzamide,
or a pharmaceutically acceptable salt thereof.

13. A compound according to claim 7,
wherein the compound is selected from the group consisting of
N-[(1S,2R)-1-Benzyl-2-hydroxy-3-(3-trifluoromethoxy-benzylamino)-propyl]-3-methyl-5-(2-thiophen-2-yl-pyrrolidine-1-carbonyl)-benzamide,
N-[(1S,2R)-1-Benzyl-2-hydroxy-3-(3-trifluoromethoxy-benzylamino)-propyl]-5,N′-dimethyl-N′-(4-methyl-thiazol-2-ylmethyl)-isophthalamide,
N-[(1S,2R)-1-Benzyl-2-hydroxy-3-(3-trifluoromethyl-benzylamino)-propyl]-3-(methanesulfonyl-methyl-amino)-5-[(R)-2-(4-methyl-thiazol-2-yl)-pyrrolidine-1-carbonyl]-benzamide,
N-[(1S,2R)-1-Benzyl-2-hydroxy-3-(3-trifluoromethyl-benzylamino)-propyl]-3-(methanesulfonyl-methyl-amino)-5-(2-phenyl-pyrrolidine-1-carbonyl)-benzamide,
N-[(1S,2R)-1-Benzyl-2-hydroxy-3-(3-trifluoromethyl-benzylamino)-propyl]-3-methyl-5-(2-phenyl-pyrrolidine-1-carbonyl)-benzamide,
N-[(1S,2R)-1-Benzyl-2-hydroxy-3-(3-trifluoromethyl-benzylamino)-propyl]-5,N′-dimethyl-N′-pyridin-2-ylmethyl-isophthalamide,
N-[(1S,2R)-1-Benzyl-2-hydroxy-3-(3-trifluoromethyl-b enzylamino)-propyl]-3-(methanesulfonyl-methyl-amino)-5-(2-thiophen-2-yl-pyrrolidine-1-carbonyl)-benzamide,
N-[(1S,2R)-1-Benzyl-2-hydroxy-3-(3-trifluoromethyl-benzylamino)-propyl]-3-methyl-5-(2-thiophen-2-yl-pyrrolidine-1-carbonyl)-benzamide,
N-[(1S,2R)-1-Benzyl-2-hydroxy-3-(3-trifluoromethyl-benzylamino)-propyl]-3-(2-p-tolyl-pyrrolidine-1-carbonyl)-benzamide, and
N-[(1S,2R)-1-Benzyl-2-hydroxy-3-(3-trifluoromethyl-benzylamino)-propyl]-3-[2-(4-methoxy-phenyl)-pyrrolidine-1-carbonyl]-benzamide,
or a pharmaceutically acceptable salt thereof.

14. A compound according to claim 7,
wherein the compound is selected from the group consisting of
N-[(1S,2R)-1-Benzyl-2-hydroxy-3-(3-trifluoromethyl-benzylamino)-propyl]-3-[2-(3-chloro-phenyl)-pyrrolidine-1-carbonyl]-benzamide,
N-[(1S,2R)-1-Benzyl-2-hydroxy-3-(3-trifluoromethyl-benzylamino)-propyl]-3-(2-m-tolyl-pyrrolidine-1-carbonyl)-benzamide,
N-[(1S,2R)-1-Benzyl-2-hydroxy-3-(3-trifluoromethyl-benzylamino)-propyl]-3-[2-(3-chloro-4-fluoro-phenyl)-pyrrolidine-1-carbonyl]-benzamide,
N-[(1S,2R)-1-Benzyl-2-hydroxy-3-(3-trifluoromethyl-benzylamino)-propyl]-3-[2-(4-chloro-3-methyl-phenyl)-pyrrolidine-1-carbonyl]-benzamide,
N-[(1S,2R)-1-Benzyl-2-hydroxy-3-(3-trifluoromethyl-benzylamino)-propyl]-3-[2-(2,4-dimethyl-phenyl)-pyrrolidine-1-carbonyl]-benzamide,
N-[(1S,2R)-1-Benzyl-2-hydroxy-3-(3-trifluoromethyl-benzylamino)-propyl]-3-[2-(4-ethyl-phenyl)-pyrrolidine-1-carbonyl]-benzamide,
N-[(1S,2R)-1-Benzyl-2-hydroxy-3-(3-trifluoromethyl-benzylamino)-propyl]-3-[2-(4-fluoro-phenyl)-3-methyl-pyrrolidine-1-carbonyl]-benzamide,
3-[4-Acetyl-2-(4-fluoro-phenyl)-pyrrolidine-1-carbonyl]-N-[(1S,2R)-1-benzyl-2-hydroxy-3-(3-trifluoromethyl-benzylamino)-propyl]-benzamide,
N-[(1S,2R)-1-Benzyl-2-hydroxy-3-(3-trifluoromethyl-benzylamino)-propyl]-3-((S)-2-benzyl-pyrrolidine-1-carbonyl)-benzamide, and
N-[(1S,2R)-1-Benzyl-2-hydroxy-3-(3-trifluoromethyl-benzylamino)-propyl]-3-((R)-2-phenyl-pyrrolidine-1-carbonyl)-benzamide,
or a pharmaceutically acceptable salt thereof.

15. A compound according to claim 7,
wherein the compound is selected from the group consisting of
N-[(1S,2R)-1-Benzyl-2-hydroxy-3-(3-trifluoromethyl-benzylamino)-propyl]-3-[(S)-2-(4-fluoro-phenyl)-pyrrolidine-1-carbonyl]-benzamide,
N-[(1S,2R)-1-Benzyl-2-hydroxy-3-(3-trifluoromethyl-benzylamino)-propyl]-3-[2-(3-fluoro-phenyl)-pyrrolidine-1-carbonyl]-benzamide,
N-[(1S,2R)-1-Benzyl-2-hydroxy-3-(3-trifluoromethyl-benzylamino)-propyl]-3-(2-cyclohexyl-pyrrolidine-1-carbonyl)-benzamide,
N-[(1S,2R)-1-Benzyl-2-hydroxy-3-(3-trifluoromethyl-benzylamino)-propyl]-3-(2-isobutyl-pyrrolidine-1-carbonyl)-benzamide,
3-(2-Benzoyl-pyrrolidine-1-carbonyl)-N-[(1S,2R)-1-benzyl-2-hydroxy-3-(3-trifluoromethyl-benzylamino)-propyl]-benzamide,
N-[(1S,2R)-1-Benzyl-2-hydroxy-3-(3-trifluoromethyl-benzylamino)-propyl]-3-(2-cyclopentyl-pyrrolidine-1-carbonyl)-benzamide, and
N-[(1S,2R)-1-Benzyl-2-hydroxy-3-(3-trifluoromethyl-benzylamino)-propyl]-3-(2-tert-butyl-pyrrolidine-1-carbonyl)-benzamide,
or a pharmaceutically acceptable salt thereof.

16. A pharmaceutical composition comprising a compound according to claim 7 and a pharmaceutically acceptable carrier and/or a pharmaceutically acceptable auxiliary substance.
